(19) United States
(12) Patent Application Publication
(10) Pub. No.: US 2005/0153287 A1
(43) Pub. Date: Jul. 14, 2005

(54) Nr3b nmda receptor subunit compositions and related methods

(76) Inventor: Yasunori Hayashi, Brookline, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/493,047

(22) PCT Filed: Oct. 18, 2002
(86) PCT No.: PCT/US02/33408

Related U.S. Application Data
(60) Provisional application No. 60/344,545, filed on Oct. 19, 2001.

(51) Int. Cl. C12Q 1/68; G01N 33/53; G01N 33/567; A01K 67/00; C07K 14/705; C07H 21/04

(52) U.S. Cl. 435/6; 435/7.2; 435/69.1; 435/320.1; 435/325; 530/350; 536/23.5; 800/8

(57) ABSTRACT

The invention relates to the identification of a new mouse gene and its trancript that encode a new NR3B subunit of NMDA receptors.

### RELATED APPLICATIONS

This application claims the benefit under 35 USC 119 of U.S. provisional application Ser. No. 60/344,545, filed Oct. 19, 2001, now pending.

### FIELD OF THE INVENTION

The present invention relates to novel NR3B genes and transcripts that have been cloned from mouse. The invention is directed to the isolated NR3B nucleic acids, the polypeptides encoded by these nucleic acids, agents that selectively bind thereto, and various diagnostic, therapeutic, and research uses of these compositions.

### BACKGROUND OF THE INVENTION

Glutamate is a major excitatory neurotransmitter in the mammalian central nervous system. Its action is mediated by two distinct classes of glutamate receptors, ionotropic and metabotropic glutamate receptors (Seeburg, 1993; Nakanishi et al., 1996; Dingledine et al., 1999). The ionotropic glutamate receptor is a ligand-gated cation channel that passes cation upon glutamate binding. It consists of several different subclasses including NMDA, AMPA, kainate, and δ, which, in turn, are hetero-oligomers of several subunits (Seeburg, 1993; Hollmann and Heinemann, 1994; Dingledine et al., 1999). The NMDA receptor is a hetero-oligomer of NR1, NR2A, NR2B, NR2C, NR2D, and NR3A (also called χ-1 or NMDAR-L) subunits. NR1 is a key subunit that confers essential functions of the NMDA receptor and is expressed ubiquitously in the central nervous system. In contrast, other subunits show more limited expression and confer a functional diversity. For example, during development of the visual cortex, NR2B is expressed predominantly in the early stages. After functional maturation of the cortex, predominant expression switches to NR2A (Quinlan et al., 1999). Concomitantly, the decay time course of the NMDA receptor-mediated synaptic current becomes faster (Carmignoto and Vicini, 1992; Philpot et al., 2001). Dark rearing of animals, which prolongs the critical period of the visual cortex, delays this conversion (Quinlan et al., 1999).

In contrast with NR2 subunits, less is known about the NR3 class of subunit. NR3A is expressed ubiquitously during development and its expression level reaches a maximum at around the first postnatal week. Thereafter, the level gradually decreases, and in adult animals, NR3A is confined to limited nuclei in the thalamus, amygdala, and nucleus of the lateral olfactory tract (Ciabarra et al., 1995; Sucher et al., 1995). The NR3A subunit binds to NR1 and NR2 and acts in a dominant-negative fashion against the NMDA receptor to reduce whole-cell current as well as single-channel conductance (Ciabarra et al., 1995; Sucher et al., 1995; Das et al., 1998; Perez-Otano et al., 2001). Consistent with this role for NR3A, mice lacking NR3A have a larger NMDA receptor-mediated current and an increased dendritic spine density in cerebrocortical neurons (Das et al., 1998), suggesting that the NR3A subunit plays a role in the development and plasticity of the central nervous system through a modulation of NMDA receptor function.

There are a number of degenerative motor neuron disorders that may be linked to aberrant NMDA receptor function. One such disorder is amyotrophic lateral sclerosis (ALS). In this progressive motor neuron disorder, motor neurons consisting of anterior horn cells of the spinal cord and upper or corticospinal motor neurons die, which results in an inexorable loss of muscle innervation and atrophy, eventually leading to death of the patient. Mutations in superoxide dismutase (SOD) have been suggested as one of the causes of ALS, but may only account for a very small population of patients (˜2%) with the disease.

The diagnosis of motor neuron diseases such as ALS is difficult and there is no available treatment to eliminate or reverse the inexorable loss of motor neuron function in this disease. Although a transgenic SOD animal is available as a model for the limited SOD-based ALS, good cell models or animal models for many motor neuron diseases such as non-SOD-based ALS are not available. If such models were available, they could foster research into the cause and physiological progression of motor neuron diseases. In addition, motor neuron disease models are needed for screening possible candidate molecules for use as much needed medications for the disease. Motor neuron disease models would also be useful for developing and testing treatment methods and could help increase the likelihood of positive outcome for patients with motor neuron disease such as ALS.

### SUMMARY OF THE INVENTION

Novel NR3B genes and transcripts that may be utilized to provide much-needed information about motor neuron diseases, including ALS, have identified and cloned from mouse. These novel sequences and the polypeptides they encode are useful for developing and utilizing diagnostic methods and therapeutic methods and for producing animal models for research into prevention and treatment of motor neuron disease. The lack of suitable animal models for many motor neuron diseases, including a model applicable for examination of most ALS etiologies, prevents better understanding of the parameters of the diseases. The identification of the novel mouse NR3B nucleic acids and polypeptides they encode can be used to generate cell and animal models of diseases, including but not limited to ALS, which are useful for characterizing motor neuron diseases.

According to one aspect of the invention, isolated nucleic acid molecules are provided. The isolated nucleic acid molecules are selected from the group consisting of (a) nucleic acid molecules which hybridize under high stringency conditions to a nucleic acid molecule having a nucleotide sequence set forth as SEQ ID NO:1, providing that no more than about 18% of the nucleotides are changed from SEQ ID NO:1, (b) nucleic acid molecules that differ from the nucleic acid molecules of (a) in codon sequence due to the degeneracy of the genetic code, and (c) complements of (a) or (b), wherein the nucleic acid molecules or complements thereof code for a mouse NR3B NMDA receptor subunit.

In some embodiments, the isolated nucleic acid molecule comprises a nucleic acid sequence set forth as: SEQ ID NO: 1.

According to another aspect of the invention, isolated nucleic acid molecule selected from the group consisting of: (a) fragment of nucleotides 1-3290 of SEQ ID NO: 1 between 24 and 3289 nucleotides in length, providing that no more than about 18% of the nucleotides are changed from SEQ ID NO:1, and (b) complements of (a) are provided.

According to another aspect of the invention expression vectors are provided. The expression vectors include the-foregoing isolated nucleic acid molecules operably linked to a promoter.

According to yet another aspect of the invention, host cells with the foregoing expression vectors are provided.

According to still another aspect of the invention, transgenic non-human animals that include the aforementioned expression vectors are provided.

According to another aspect of the invention, isolated polypeptides encoded by the aforementioned isolated nucleic acid molecule are provided. In some embodiments, the isolated polypeptide includes an amino acid sequence selected from the group consisting of SEQ ID NO: 2 and a fragment or functional variant of SEQ ID NO:2.

According to another aspect of the invention, binding polypeptides that selectively bind the aforementioned isolated polypeptide are provided, and the binding polypeptide is an antibody or antigen-binding fragment thereof. In some embodiments, the binding polypeptide selectively binds the polypeptide sequence of SEQ ID NO: 2.

According to yet another aspect of the invention, compositions the include a molecule selected from the group consisting of: (a) the nucleic acid of any of claims 1-3, (b) the polypeptide encoded by the isolated nucleic acid molecule of any of claims 1-3, and (c) the binding polypeptide of any of claims 9-10, and a pharmaceutically acceptable carrier are provided.

According to another aspect of the invention, methods for making medicaments are provided. The methods include placing in a pharmaceutically acceptable carrier, a molecule selected from the group consisting of: (a) the isolated nucleic acid molecules of any of claims 1-3, (b) the isolated polypeptide of any of claims 7-8, and (c) the binding polypeptides of any of claims 9-10. Some embodiments of the methods also include the step of placing comprises placing a therapeutically effective amount of the molecule selected from the group in the pharmaceutically acceptable carrier to form one or more doses.

According to another aspect of the invention methods of making a glutamate receptor in vitro are provided. The methods include introducing glutamate receptor nucleic acids into a cell, wherein the glutamate receptor nucleic acids encode a NR3B polypeptide. In some embodiments of the method the NR3B subunit is encoded by the nucleotide sequence set forth as SEQ ID NO: 1.

According to another aspect of the invention, methods for diagnosing a motor neuron disorder characterized by aberrant expression of a NR3B molecule are provided. The -methods include: detecting expression of a NR3B molecule in a first biological sample obtained from a subject, wherein a difference in expression level of the NR3B molecule compared to expression level a NR3B molecule in a control sample indicates that the subject has a motor neuron disorder characterized by aberrant expression of a NR3B molecule. In some embodiments, the methods also include detecting expression of a NR3B molecule in a second biological sample obtained from the subject at a time subsequent to the first biological sample, and comparing the expression of the NR3B molecule in the first biological sample and the second biological sample as an indication of the onset, progression, or regression of the motor neuron disorder.

In some embodiments, a decrease in expression level of the NR3B in the second biological sample compared to the expression level in the NR3B in the first biological sample indicates progression of the motor neuron disorder characterized by aberrant expression of NR3B. In other embodiments, an increase in expression level of the NR3B in the second biological sample compared to the expression level in the NR3B in the first biological sample indicates regression of the motor neuron disorder characterized by aberrant expression of NR3B. In preferred embodiments, the motor neuron disorder is amyotrophic lateral sclerosis (ALS). In some embodiments, the motor neuron disorder characterized by aberrant expression of a NR3B molecule is amyotrophic lateral sclerosis (ALS). In certain embodiments, the methods include detecting expression of a NR3B nucleic acid molecule. In some embodiments, the NR3B nucleic acid molecule comprises the nucleic acid sequence set forth as SEQ ID NO: 1 or fragment thereof. In certain embodiments, the methods include detecting expression of a NR3B polypeptide. In certain embodiments, the NR3B polypeptide is set forth as SEQ ID NO: 2 or fragment thereof. In some embodiments, detecting comprises contacting the biological sample with an agent that selectively binds the NR3B molecule. In some embodiments, the NR3B molecule is a nucleic acid and wherein the agent that selectively binds the NR3B molecule is a nucleic acid selected from the group of nucleic acid molecules comprising the nucleotide sequences that hybridize to SEQ ID NO: 1 under high stringency conditions. In certain embodiments, the NR3B molecule is a polypeptide and wherein the agent that selectively binds the NR3B molecule is a binding polypeptide selected from the group of binding polypeptides that selectively bind to SEQ ID NO: 2. In some embodiments, the biological sample is selected from the group consisting of: a neuronal cell, neuronal tissue, and spinal fluid.

According to another aspect of the invention, methods for evaluating the effect of candidate pharmacological compounds on expression of an NR3B subunit of a glutamate receptor are provided. The methods include administering a candidate pharmaceutical agent to a subject; determining the effect of the candidate pharmaceutical agent on the expression level of NR3B relative to the expression level of NR3B in a subject to which no candidate pharmaceutical agent is administered, wherein a relative increase or relative decrease in the expression level of NR3B indicates the effect of the candidate pharmaceutical compound on the expression of the NR3B subunit of the glutamate receptor. In preferred embodiments, the subject is a mouse. In preferred embodiments, the glutamate receptor is an NMDA receptor.

According to yet another aspect of the invention, methods for evaluating the effect of candidate pharmacological compounds on the expression of a NR3B subunit of a glutamate receptor are provided. The methods include contacting a candidate pharmaceutical agent with a NR3B subunit expressing cell or tissue sample; determining the effect of the candidate pharmaceutical agent on the expression level of NR3B relative to the expression level of NR3B in a NR3B subunit expressing cell or tissue sample not contacted with the candidate pharmaceutical agent, wherein a relative increase or relative decrease in the expression level of NR3B indicates the effect of the candidate pharmaceutical compound on the expression of NR3B subunit of a glutamate receptor. In some embodiments, the glutamate receptor sample is in culture. In preferred embodiments, the glutamate receptor sample is an NMDA receptor sample.

According to another aspect of the invention, methods for diagnosing a motor neuron disorder characterized by aberrant function of a NR3B molecule are provided. The methods include detecting function of a NR3B molecule in a first biological sample obtained from a subject, wherein a difference in function of the NR3B molecule compared to a NR3B molecule in a control sample indicates that the subject has a motor neuron disorder characterized by aberrant function of a NR3B molecule. Some embodiments also include: detecting function of a NR3B molecule in a second biological sample obtained from the subject at a time subsequent to the first biological sample, and comparing the function of the NR3B molecule in the first biological sample and the second biological sample as an indication of the onset, progression, or regression of the motor neuron disorder. In some embodiments, a decrease in function level of the NR3B in the second biological sample compared to the function level in the NR3B in the first biological sample indicates progression of the motor neuron disorder characterized by aberrant function of NR3B. In preferred embodiments, the motor neuron disorder is amyotrophic lateral sclerosis (ALS). In certain embodiments, an increase in function level of the NR3B in the second biological sample compared to the function level in the NR3B in the first biological sample indicates regression of the motor neuron disorder characterized by aberrant expression of NR3B.

In some embodiments, the motor neuron disorder characterized by aberrant function of a NR3B molecule is amyotrophic lateral sclerosis (ALS). Some embodiments include comprising detecting function of a NR3B nucleic acid molecule. In certain embodiments, the NR3B nucleic acid molecule comprises a nucleic acid sequence set forth as SEQ ID NO: 1 or fragment thereof. Some embodiments include detecting function of a NR3B polypeptide. In certain embodiments, the NR3B polypeptide is set forth as SEQ ID NO: 2 or fragment thereof. In some embodiments, detecting includes determining the cation passage through an NMDA receptor channel. In certain embodiments, the cation flux is determined with a method selected from the group consisting of: electrophysiological recording, drug screening assays, and ion-flux measurement. In some embodiments, the biological sample is selected from the group consisting of: a neuronal cell, neuronal tissue, and spinal fluid.

According to still another aspect of the invention, methods for evaluating the effect of candidate pharmacological compounds on function of an NR3B subunit of a glutamate receptor are provided. The methods include administering a candidate pharmaceutical agent to a subject that expresses a glutamate receptor containing a functional NR3B subunit; detecting the function of the NR3B subunit of the glutamate receptor, determining the effect of the candidate pharmaceutical agent on the function level of NR3B relative to the function level of NR3B in a subject to which no candidate pharmaceutical agent is administered, wherein a relative increase or relative decrease in the function level of NR3B indicates the effect of the candidate pharmacological compound on the function of the NR3B subunit of the glutamate receptor. In preferred embodiments, the subject is mouse. In preferred embodiments, the glutamate receptor is an NMDA receptor. In some embodiments, detecting includes determining the cation passage through an NMDA receptor channel. In certain embodiments, the cation flux is determined with a method selected from the group consisting of: electrophysiological recording, drug screening assays, and ion-flux measurement.

According to another aspect of the invention, methods for evaluating the effect of candidate pharmacological compounds on function of a NR3B subunit of a glutamate receptor are provided. The methods include contacting a glutamate receptor sample with a candidate pharmaceutical agent; detecting the function of the NR3B subunit of the glutamate receptor, determining the effect of the candidate pharmaceutical agent on the function level of NR3B relative to the function level of NR3B in a glutamate receptor sample not contacted with the candidate pharmaceutical agent, wherein a relative increase or relative decrease in the function level of NR3B indicates the effect of the candidate pharmacological agent on the function of NR3B subunit of the glutamate receptors. In some embodiments, the glutamate receptor sample is in culture. In some embodiments, the glutamate receptor sample is an NMDA receptor sample. In certain embodiments, detecting includes determining the cation passage through an NMDA receptor channel. In some embodiments, the cation flux is determined with a method selected from the group consisting of: electrophysiological recording, drug screening assays, and ion-flux measurement.

According to yet another aspect of the invention, kits for diagnosing a motor neuron disorder associated with aberrant expression of a NR3B molecule are provided. The kits include one or more nucleic acid molecules that hybridize to a NR3B nucleic acid molecule under high stringency conditions and instructions for the use of the nucleic acid molecules in the diagnosis of a motor neuron disorder associated with aberrant expression of a NR3B molecule. In some embodiments, the one or more nucleic acid molecules are a first primer and a second primer and, wherein the first primer and the second primer are constructed and arranged to selectively amplify at least a portion of an isolated NR3B nucleic acid molecule comprising SEQ ID NO: 1.

According to another aspect of the invention, kits for diagnosing a NR3B-associated motor neuron disorder in a subject are provided. The kits include one or more binding polypeptides that selectively bind to a NR3B polypeptide, and instructions for the use of the binding polypeptides in the diagnosis of a motor neuron disorder associated with aberrant expression of a NR3B molecule. In some embodiments, the one or more binding polypeptides are antibodies or antigen-binding fragments thereof. In certain embodiments, the NR3B polypeptide is encoded by a nucleic acid comprising a nucleotide sequence set forth as SEQ ID NO:1.

According to another aspect of the invention, methods for treating a subject with a motor neuron disorder characterized by decreased expression of a NR3B molecule are provided. The methods include administering to the subject an amount of a NR3B nucleic acid molecule effective to increase expression of a NR3B polypeptide and treat the motor neuron disorder.

According to yet another aspect of the invention, methods for treating a subject with a motor neuron disorder characterized by decreased expression of a NR3B polypeptide are provided. The methods include-administering to the subject an amount of a NR3B polypeptide effective to treat the motor neuron disorder.

According to yet another aspect of the invention, methods for treating a subject with a motor neuron disorder characterized by increased expression of a NR3B nucleic acid molecule are provided. The methods include administering to the subject an amount of an antisense molecule to a NR3B nucleic acid molecule effective to treat the motor neuron disorder.

According to yet another aspect of the invention, methods for treating a subject with a motor neuron disorder characterized by increased expression of a NR3B polypeptide are provided. The methods include administering to the subject an amount of a NR3B polypeptide binding polypeptide effective to treat the motor neuron disorder. In some embodiments, the binding polypeptide agent is an antibody or an antigen-binding fragment thereof

According to another aspect of the invention, methods for treating a subject with a motor neuron disorder characterized by decreased function of a NR3B molecule are provided. The methods include administering to the subject an amount of a NR3B nucleic acid molecule effective to treat the motor neuron disorder.

According to another aspect of the invention, methods for treating a subject with a motor neuron disorder characterized by decreased function of a NR3B polypeptide are provided. The methods include administering to the subject an amount of a NR3B polypeptide effective to treat the motor neuron disorder.

According to yet another aspect of the invention, methods for treating a subject with a motor neuron disorder characterized by increased function of a NR3B nucleic acid molecule are provided. The methods include administering to the subject an amount of an antisense molecule to a NR3B nucleic acid molecule effective to treat the motor neuron disorder.

According to still another aspect of the invention, methods for treating a subject with a motor neuron disorder characterized by increased function of a NR3B polypeptide are provided. The methods include administering to the subject an amount of a NR3B polypeptide binding polypeptide effective to treat the motor neuron disorder.. In some embodiments, the binding polypeptide agent is an antibody or an antigen-binding fragment thereof.

According to another aspect of the invention, methods for producing a NR3B polypeptide or fragment-thereof are provided. The methods include providing an isolated NR3B nucleic acid molecule operably linked to a promoter, wherein the NR3B nucleic acid molecule encodes the NR3B polypeptide or fragment thereof, and expressing the NR3B nucleic acid molecule in an expression system. In some embodiments, the methods also include isolating the NR3B polypeptide or a fragment thereof from the expression system. In some embodiments, the NR3B nucleic acid molecule is set forth as SEQ ID NO: 1.

According to another aspect of the invention, methods for making a NR3B polypeptide are provided. The methods include culturing the host cell of claim 5, and isolating the NR3B polypeptide from the culture.

According to another aspect of the invention, methods for preparing a model of a motor neuron disease characterized by aberrant expression of a NR3B molecule are provided. The methods include introducing into a cell, a NR3B molecule. In some embodiments, the motor neuron disorder is Amyotrophic Lateral Sclerosis (ALS). In some embodiments, the NR3B molecule is a NR3B nucleic acid molecule set forth in SEQ ID NO: 1. In certain embodiments, the NR3B molecule is a NR3B polypeptide set forth in SEQ ID NO: 2. In some embodiments, the cell is in a non-human animal subject. In some embodiments, the model is a knock-out model.

According to another aspect of the invention, methods for preparing an animal model of a motor neuron disorder characterized by aberrant function of a NR3B molecule are provided. The methods include introducing into a non-human subject, an aberrant NR3B molecule; and detecting expression of the aberrant NR3B molecule in a first biological sample obtained from the non-human subject. In some embodiments, the aberrant NR3B molecule is not functional. In certain embodiments, the aberrant NR3B molecule has increased function level compared to a control NR3B function level. In some embodiments, the aberrant NR3B molecule has decreased function level compared to a control NR3B function level. In preferred embodiments, the motor neuron disorder is Amyotrophic Lateral Sclerosis (ALS). In certain embodiments, the NR3B molecule is a NR3B nucleic acid molecule. In some embodiments, the NR3B molecule is a NR3B polypeptide.

According to another aspect of the invention, methods for preparing a non-human animal model of a motor neuron disorder characterized by reduced expression of a NR3B molecule are provided. The methods include administering to a non-human subject an effective amount of an anti-sense molecule to a NR3B nucleic acid molecule to reduce expression of the NR3B nucleic molecule in the non-human subject. In some embodiments, the NR3B molecule is a nucleic acid molecule selected from the group containing SEQ ID NO: 1 and SEQ ID NO:3.

According to yet another aspect of the invention, methods for preparing a non-human animal model of a motor neuron disorder characterized by reduced expression of a NR3B molecule are provided. The methods include administering to a non-human subject an effective amount of a binding polypeptide to a NR3B polypeptide to reduce expression of the NR3B polypeptide in the non-human subject. In some embodiments, the binding polypeptide agent is an antibody or an antigen-binding fragment thereof. In certain embodiments, the NR3B molecule a nucleic acid molecule selected from the group containing SEQ ID NO: 1 and SEQ ID NO:3.

All of the foregoing aspects of the invention relate to the motor neuron disorders and diseases described herein.

### BRIEF DESCRIPTION OF THE DRAWINGS

The descriptions include drawings that may reference color components. The figures are illustrative only and are not required for enablement of the invention disclosed herein.

FIGS. 1A-1D show digital images relating the structure of the glutamate receptor subtype NR3B. (A) Phylogenic tree of the members of the glutamate receptor family based on the neighbor joining method. The horizontal length of the branch indicates the distance between polypeptides. (B) Mouse and human NR3B sequences (SEQ ID NO:3 and SEQ ID NO:4 amino acids 1-890). The human sequence is not complete at the C-terminus. Identical amino acids between two species are indicated by “:” and homologous ones by ”.”. SP and M1˜M4 indicate the predicted signal peptide and membrane-associated region, respectively. Putative glycosylation sites are boxed. Upward arrowheads indicate exon boundaries. Amino acids implicated in ligand binding of GluR2 from a crystallographic study are shown below the mouse sequence at the corresponding positions. (C) A comparison of the M2 domain that forms channel pore (GluR1, GluR2, GluR5, KA-1, δ2, NR1, NR2A, NR3A, NR3B are SEQ ID NOs:15-23 respectively). The critical amino acid at the Q/R/N site, which controls ion permeability and rectification, is glycine followed by an arginine in both NR3B and NR3A (shown under arrow). In human NR3B, the Q/R/N site is an arginine (B). (D) Genomic structure of mouse NR3B. Open and shaded boxes are coding and non-coding regions, respectively. The 5′-non-coding region may extend further upstream.

FIG. 2A-2D contain digital photomicrographic images depicting tissue distribution of mouse NR3B. FIG. 2A is a gel demonstrating NR3B transcripts detected by RT-PCR with primers that span exons 1 and 2. The PCR product of the expected length was detected in the brainstem (BS) and spinal cord (Sp) and, to a much lesser extent, in the cerebellum (Cb). When reverse transcriptase was omitted, (-RT), the product was not detected. As a positive control for RT-PCR, the NR1 was amplified in parallel, producing two bands that correspond to different splice variants. OB, olfactory bulb; Cx, cerebral cortex; Str, striatum; Hip, hippocampus; Th-MB, thalamus to midbrain region. FIG. 2B depicts Northern analysis of mouse spinal cord polyA+RNA, which detected a single band at around 3.5 kb. FIG. 2C shows In situ hybridization of sagittal sections of mouse brain with ³³P-labeled antisense (AS) and sense (S) probes. The antisense probe detected a discrete signal in trigeminal motor (V), facial (VIII), and ambiguous nuclei (IX). The cerebellum also had a faint signal. FIG. 2D shows higher magnification of cranial nerve nuclei. A restricted expression of NR3B was detected in motor neurons that control somatic movement but not in those controlling ocular movement. Left: low and high magnification images of Nissl-stained sections of oculomotor (III), trochlear (IV), trigeminal motor (V), facial (VII) and abducens (VI) nuclei are shown. Right: In situ hybridization using anti-sense and sense probes labeled with digoxigenin. Strong signal was observed in trigeminal motor and facial nuclei but not of those innervating extraocular muscles. Bars, 1 mm for C and 100 μm for D.

FIG. 3A, 3B shows digitized photomicrographic images of expression of NR3B in spinal cord. Left panel: low-magnification images of sections stained with Nissl and adjacent sections hybridized with antisense probe. Right panel: higher magnification of the same sections. Somatic motor neurons are strongly labeled in C2 and L2 levels. In contrast, the expression of NR3B was significantly weaker in the motor neurons at the L6 level. Surrounding these cells, there was a plexus of serotonin immunoreactive fibers (arrow heads), which is one of characteristics of motor neurons in Onuf's nucleus. Bars, 100 μm.

FIG. 4A-4D contain digitized images of glutamate-induced whole-cell current recorded from HEK293 cells expressing NR3B. NR3B was coexpressed with the NR1 and NR2A subunits. FIG. 4A shows sample electrophysiological traces of glutamate-induced current recorded at −80 mV to +60 mV (20 mV step) in the presence or absence of 1 mM Mg²⁺. FIG. 4B shows the remaining current in NR3B-expressing cells exhibits a Mg²⁺ block indistinguishable from cells not expressing NR3B. FIG. 4C shows NR3B3 acts as a dominant-negative subunit and suppresses glutamate-induced whole-cell current in cells coexpressing NR1 and NR2A. The distribution of averaged responses obtained at +60 mV in Mg²⁺-free solution is shown in a cumulative plot. Increasing the amount of NR3B plasmid (0, 1, and 3 μg) versus other subunits (each 1 μg) caused a concomitant decrease in current amplitude. AMPA receptor mediated current was unchanged by the coexpression with NR3B. FIG. 4D illustrates the coexpression with NR3B did not change the expression levels of NR1 or NR2A. NR1 and NR2A tagged with GFP were individually expressed with NR3B and their expression levels were measured by the fluorescence intensity. The statistical significance in this figure was assessed by Kolmogorov-Smirnov test.

### DETAILED DESCRIPTION OF THE INVENTION

The present invention, in one aspect, involves the cloning of CDNAs that encode a mouse and human NR3B polypeptide. The sequence of the coding portion of the mouse gene is presented as SEQ ID NO: 1, and the predicted amino acid sequence of this gene's polypeptide product is presented as SEQ ID NO: 2. The sequence of the coding portion of the human gene is presented as SEQ ID NO: 3, and the predicted amino acid sequence of this gene's polypeptide product is presented as SEQ ID NO: 4.

Sequence analysis revealed that the mouse NR3B polypeptide shows 51% similarity to the NR3A subunit of the NMDA receptor. The invention thus involves in one aspect mouse NR3B polypeptides, nucleic acid molecules encoding those polypeptides, functional modifications and variants of the foregoing, useful fragments of the foregoing, as well as therapeutic and diagnostic products (including antibodies), non-human animal models, and methods relating thereto.

As used herein, the term “NR3B” means mouse NR3B and the term “hNR3B” means human NR3B. As used herein, the term “NR3B polypeptide” refers to polypeptides/proteins with a deduced structure with the same general characteristics as a glutamate receptor subunit with an N-terminal signal peptide and four membrane-associated regions. Five (in mouse) or four (in human) consensus sequences for N-glycosylation sites are found on the N-terminal domain and one (for both species) at the loop between the third and fourth membrane-associated regions. The intracellular C-terminus of mouse NR3B has three threonines and five serines, which may serve as regulatory phosphorylation sites. The C-terminus ends with Ala-Glu-Ser, which does not conform to the consensus PDZ domain protein-binding site sequence typical of other glutamate receptor family members (Songyang et al., 1997; Sheng and Sala, 2001).

As used herein, a subject is a human, non-human primate, cow, horse, pig, sheep, goat, dog, cat, or rodent, including but not limited to: guinea pig, rat, and mouse. As used herein an animal is a non-human primate, cow, horse, pig, sheep, goat, dog, cat, or rodent, including, but not limited to: guinea pig, rat, and mouse. In all embodiments, human and mouse NR3B molecules and human and mouse subjects are preferred.

As used herein, the term “NR3B-associated motor neuron disorder” means any motor neuron disorder or motor neuron disease characterized by aberrant expression of NR3B. Motor neuron diseases and disorders may include, but are not limited to: Amyotrophic lateral sclerosis (ALS): hereditary ALS, also called Lou Gehrig's disease or Maladie de Charcot (e.g.: dominant, recessive, neurofilament heavy subunit: 22q12, superoxide dismutase: 21q22.1, childhood-onset ALS, ALS2, ALS4, ALS5); ALS with bulbar onset; benign course and bunina bodies; Sporadic Amyotrophic Lateral Sclerosis; Primary Lateral Sclerosis; Primary Muscular Atrophy (PMA); Western Pacific ALS; Western Pacific ALS-like disorders; Insulinoma; Monomelic Amyotrophy; Bulbar Syndromes (e.g. Brown-Vialetto-van Laere, Fazio-Londe, Congenital Bulbar Syndrome, Kennedy's Syndrome, Madras motor neuron disease, Spino-bulbar Muscular Atrophy, Worster-Drought syndrome, Congenital Suprabulbar Paralysis); Sporadic Bulbo-spinal Muscular Atrophy; Motor Neuropathy, (e.g. distal: GM1 or GalNAc-GDla antibody and multifocal (MMN): GM1 antibody); myopathies (e.g. Paraneoplastic Motor Neuropathy; Paraneoplastic Lower Motor Neuron syndrome; Poliomyelitis and Post-polio syndrome; Diabetic Amyotrophy; Acute Axonal Motor Neuropathy, Porphyria); Spinal Muscular Atrophy (SMA) [e.g. hereditary, SMN (5q), androgen receptor, bulbar SMA, distal, hexosaminidase A (Tay-Sachs), HMN 1, H 2, HMN, HMN 5B, HMN 7 (vocal cord), HMN J, ulnar-median, diaphragmatic paralysis and neonatal, SPG 14, upper limb predominance, dominant, proximal SMA, benign congenital with contractures, congenital with leg weakness, MSN-P (Okinawa type), Scapuloperoneal syndromes, Congenital with Arthrogryposis, Werdnig-Hoffman, Kugelberg-Welander, Spinal Muscular Atrophy 2 (SMA2), X-linked Infant SMA and artirogryposis, and toxic]; Primary Lateral Sclerosis2; Atypical Motor Neuron diseases with ophthalnoplegia and extrapyramidal disorders4; multiple systems atrophy, sporadic or autosomal dominant; striatonigral degeneration; Adult-onset sporadic olivopontocerebellar atrophy (OPCA); Shy-Drager syndrome; Polyglucosan Body disease; motor neuronopathy with cataracts and skeletal abnormalities; Disinhibition-dementia-Parkinsonism-amyotrophy complex (DDPAC); neuropathies (e.g. myopathies: distal; Myotonic Dystrophy; Inclusion body myositis; Myasthenia Gravis; peripheral nerve lesion; median: recurrent motor branch, anterior interosseus, ulnar: guyon canal, radial: posterior interosseus, brachial plexopathy, bulbar involvement, Bulbo-spinal Muscular Atrophy, androgen receptor: X-linked; toxic: (e.g. lead; dapsone; botulism; tick paralysis); infections (e.g. Polio, Central European encephalitis, Creutzfeld-Jacob); Amyotrophy; Polyneuropathy (±Demyelinating); Multifocal Motor Neuropathy; (MMN); Hopkins' Syndrome (e.g. acute post-asthmatic amyotrophy); Hirayama's disease; O'Sullivan-McLeod syndrome (e.g. slow progression); Gowers; Machado-Joseph; and Arthrogryposis-lower motor neuron disease.

As used herein, the term “aberrant” refers to decreased expression (including zero expression) or increased expression of the natural NR3B molecule (nucleic acid or polypeptide) as compared to its expression in a subject who does not have a NR3B-associated disorder. Aberrant expression can also refer to increased expression of a mutant NR3B molecule (nucleic acid or polypeptide) as compared to its expression in a subject who does not have a NR3B-associated disorder. For example, the aberrant expression is expression that is not about 100% of the level of NR3B in a subject free of a NR3B-associated disorder. In another example, the level of NR3B expression could be outside of the range of expected levels in normal subjects. Aberrant expression may be determined by comparing levels of NR3B molecules to those levels in controls. The control(s) include positive and negative controls which may be a predetermined value that can take a variety of forms. The control(s) can be a single cut-off value, such as a median or mean, or can be established based upon comparative groups, such as in groups having normal amounts of NR3B and groups having abnormal amounts of NR3B molecules.

As used herein, a compound or signal that “modulates the activity of an NMDA receptor” refers to a compound or signal that alters the activity of NMDA receptors so that activity of the NMDA receptor is different in the presence of the compound or signal than in the absence of the compound or signal. In particular, such compounds or signals include agonists and antagonists. The term agonist refers to a substance or signal, such as NMDA, that activates receptor function; and the term antagonist refers to a substance that interferes with receptor function. Typically, the effect of an antagonist is observed as a blocking of activation by an agonist. Antagonists include competitive and non-competitive antagonists. A competitive antagonist (or competitive blocker) interacts with or near the site specific for the agonist (e.g., ligand or neurotransmitter). A non-competitive antagonist or blocker inactivates the functioning of the receptor by interacting with a site other than the site that interacts with the agonist.

As understood by those of skill in the art, assay methods for identifying compounds that modulate NMDA receptor activity (e.g., agonists and antagonists) generally require comparison to a control. One type of a “control” cell or “control” culture is a cell or culture that is treated substantially the same as the cell or culture exposed to the test compound, except the control culture is not exposed to test compound. For example, in methods that use voltage clamp electrophysiological procedures, the same cell can be tested in the presence and absence of test compound, by merely changing the external solution bathing the cell. Another type of “control” cell or “control” culture may be a cell or a culture of cells which is identical to the transfected cells, except the cells employed for the control culture do not express functional NR3B subunits. In this situation, the response of test cell to test compound is compared to the response (or lack of response) of receptor-negative (control) cell to test compound, when cells or cultures of each type of cell are exposed to substantially the same reaction conditions in the presence of compound being assayed.

Another example of a comparative group is a group having a particular disease, condition and/or symptoms and a group without the disease, condition and/or symptoms.

Another comparative group is a group with a family history of a particular disease and a group without such a family history of the particular disease. The predetermined control value can be arranged, for example, where a tested population is divided into groups, such as a low-risk group, a medium-risk group and a high-risk group or into quadrants or quintiles, the lowest quadrant or quintile being individuals with the lowest risk or highest expression levels of NR3B and the highest quadrant or quintile being individuals with the highest risk or lowest expression levels of NR3B molecules.

The predetermined value of a control will depend upon the particular population selected. For example, an apparently healthy population will have a different “normal” NR3B expression level range than will a population which is known to have a condition characterized by aberrant NR3B expression. Accordingly, the predetermined value selected may take into account the category in which an individual falls. Appropriate ranges and categories can be selected with no more than routine experimentation by those of ordinary skill in the art. By “abnormally high” it is meant high relative to a selected control. Typically the control will be based on apparently healthy individuals in an appropriate age bracket or based on normally expressed NMDA receptors.

It will also be understood that the controls according to the invention may be, in addition to predetermined values, samples of materials tested in parallel with the experimental materials. Examples include samples from control populations or control samples generated through manufacture to be tested in parallel with the experimental samples.

According to one aspect of the invention, an isolated nucleic acid molecule is provided. The isolated nucleic acid molecule is selected from the group consisting of: (a) nucleic acid molecules which hybridize under high stringency conditions to a nucleic acid molecule having a nucleotide sequence set forth as SEQ ID NO: 1 and that code for a NR3B polypeptide, (b) nucleic acid molecules that differ from the nucleic acid molecules of (a) in codon sequence due to the degeneracy of the genetic code, and (c) complements of (a), no more than about 18% of the nucleotides in (a) are changed from SEQ ID NO: 1, and the sequences exclude nucleic acids having nucleotide sequences that are contained within SEQ ID NO: 1 and that are known as of the filing date of this application. Preferably, no more than about 17%, 16%, 15%, 14%, 13%, 12%, 11%, 10%, 9%, 8%, 7%, 6%, 5%, 4%, 3%, 2%, or 1% of the nucleotides are changed relative to SEQ ID NO:1 in the nucleic acid molecules that hybridize under stringent conditions to SEQ ID NO:1.

The preferred isolated nucleic acids of the invention are NR3B nucleic acid molecules which encode a NR3B polypeptide. As used herein, a NR3B polypeptide refers to a polypeptide that is encoded by a nucleic acid including the nucleotide sequence set forth as SEQ ID NO: 1, or a functional fragment thereof, or a finctional equivalent thereof (e.g., a nucleic acid sequence encoding the same polypeptide as encoded by SEQ ID NO: 1), provided that the finctional fragment or equivalent encodes a polypeptide which exhibits a NR3B functional activity. As used herein, a NR3B functional activity refers to the ability of a NR3B polypeptide to modulate one or more of the following parameters: NMDA receptor activity, cation passage, and/or NMDA ligand binding. Although not wishing to be bound to any particular theory or mechanism, it is believed that the NR3B polypeptide may affect at least some of the above-noted cell functions by participating in the varied subunit control of glutamate receptors such as the NMDA receptor in human and animal cells. NR3B functional activity can be determined, for example, by measuring the ability of NMDA receptors to pass cations upon activation with ligand. Such measurement can be performed by electrophysiological recording coupled with application of ligand, agonists, antagonists, and control chemicals, using procedures understood by those of skill in the art. (See, for example, the electrophysiological assay described in the Examples).

In preferred embodiments, the isolated NR3B nucleic acid molecule is SEQ ID NO: 1 and the isolated NR3B polypeptide is SEQ ID NO: 2.

The invention provides isolated nucleic acid molecules which code for NR3B polypeptides and which hybridize under high stringency conditions to a nucleic acid molecule consisting of the nucleotide set forth in SEQ ID NO: 1. Such nucleic acids may be DNA, RNA, composed of mixed deoxyribonucleotides and ribonucleotides, or may incorporate synthetic non-natural nucleotides. Preferably this group of sequences excludes hNR3B sequences. Various methods for determining the expression of a nucleic acid and/or a polypeptide in cells are known to those of skill in the art and are described further below and in the Examples. As used herein, the term polypeptide is meant to include large molecular weight proteins and polypeptides and low molecular weight polypeptides or fragments thereof.

The term “high stringency conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin (BSA), 2.5 mM NaH₂PO₄ (pH 7), 0.5% SDS, 2 mM EDTA). SSC is 0.15M sodium chloride/0.015M sodium citrate, pH 7; SDS is sodium dodecyl sulphate; and EDTA is ethylenediaminetetracetic acid. After hybridization, the membrane upon which the DNA is transferred is washed at 2×SSC at room temperature, and then at 0.1×SSC/0.1×SDS at temperatures up to 68° C.

The foregoing set of hybridization conditions is but one example of high stringency hybridization conditions known to one of ordinary skill in the art. There are other conditions, reagents, and so forth which can be used, which result in a high stringency hybridization. The skilled artisan will be familiar with such conditions, and thus they are not given here. It will be understood, however, that the skilled artisan will be able to manipulate the conditions in a manner to permit the clear identification of homologs and alleles of NR3B nucleic acid molecules of the invention. The skilled artisan also is familiar with the methodology for screening cells and libraries for expression of such molecules which then are routinely isolated, followed by isolation of the pertinent nucleic acid molecule and sequencing.

In general homologs and alleles typically will share at least 80% nucleotide identity and/or at least 80% amino acid identity to SEQ ID NO: 1 and SEQ ID NO: 2, respectively, in some instances will share at least 81%, 82%, 83%, 84%, 85%, 86%, 87%, 88%, 89%, 90%, 91%, 92%, 93%, 94%, 95%, 96%, 97%, 98%, or 99, 99.5% nucleotide identity and/or at least 81%, 82%, 83%, 84%, 85%, 86%, 87%, 88%, 89%, 90%, 91%, 92%, 93%, 94%, 95%, 96%, 97%, 98%, or 99, 99.5% amino acid identity. The percent identity can be calculated using various publicly available software tools developed by NCBI (Bethesda, Md.) that can be obtained through the internet (ftp:/ncbi.nlm.nih.gov/pub/). Exemplary tools include the BLAST system available at http://www.ncbi.nlm.nih.gov, which uses algorithms developed by Altschul et al. (Nucleic Acids Res. 25:3389-3402, 1997). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acid molecules also are embraced by the invention.

In screening for NR3B genes, a Southern blot may be performed using the foregoing conditions, together with a detectably labeled probe (e.g. radioactive or chemiluminescent probes). After washing the membrane to which the DNA is finally transferred, the membrane can be placed against X-ray film or a phosphorimager to detect the radioactive or chemiluminescent signal. In screening for the expression of NR3B RNA, Northern blot hybridizations using the foregoing conditions can be performed on samples taken from subjects suspected of having a condition characterized by aberrant expression of a NR3B molecule, e.g., abnormal NMDA receptor function and/or abnormal NR3B polypeptide expression. Amplification protocols such as PCR using primers that hybridize to the sequences presented also can be used for detection of the NR3B genes or expression thereof

Identification of related sequences can be achieved using PCR and other amplification techniques suitable for cloning related nucleic acid sequences. Preferably, PCR primers are selected to amplify portions of a nucleic acid sequence believed to be conserved (e.g., a channel pore conformation domain, a ligand binding domain, etc.). Again, nucleic acids are preferably amplified from a tissue-specific library (e.g., neuronal tissue such as spinal cord and brain). One-also can use expression cloning-utilizing the antisera described herein-to identify nucleic acids that encode related proteins.

The invention also includes degenerate nucleic acid molecules which include alternative codons to those present in the native materials. For example, serine residues are encoded by the codons TCA, AGT, TCC, TCG, TCT, and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating NR3B protein. Similarly, nucleotide sequence triplets that encode other amino acid residues include, but are not limited to: CCA, CCC, CCG, and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

According to another aspect of the invention, further isolated nucleic acid molecules that are based on the above-noted NR3B nucleic acid molecules are provided. In this aspect, the isolated nucleic acid molecules are selected from the group that consists of (a) a fragment of the nucleotide sequence set forth as SEQ ID NO: 1 between 24 and 32 nucleotides in length or more, and (b) complements of (a).

The invention also provides isolated fragments of SEQ ID NO: 1 or complements of SEQ ID NO: 1. A fragment is one that is a ‘signature’ for the larger nucleic acid. It, for example, is long enough to assure that its precise sequence is not found in molecules outside of the NR3B nucleic acid molecules defined above. Those of ordinary skill in the art may apply no more than routine procedures to determine if a fragment is within the human or mouse genome. Fragments, however, exclude fragments completely composed of the nucleotide sequences that are contained within SEQ ID NO: 1 and that are known as of the filing date of this application.

Fragments can be used as probes in Southern blot, Northern blot, and Gene Chip/microarray assays to identify such nucleic acid molecules, or can be used in amplification assays such as those employing PCR As known to those skilled in the art, large probes such as 200 nucleotides or more are preferred for certain uses such as Southern blots, while smaller fragments will be preferred for uses such as in PCR and gene chip/microarray assays. Fragments also can be used to produce fusion proteins for generating antibodies or determining binding of the polypeptide fragments, or for generating immunoassay components. Likewise, fragments can be employed to produce nonfused fragments of the NR3B polypeptides that are useful, for example, in the preparation of antibodies in immunoassays. Fragments further can be used as antisense molecules to inhibit the expression of NR3B nucleic acids and polypeptides, particularly for therapeutic purposes as described in greater detail below.

As will be recognized by those skilled in the art, the size of the fragment will depend upon its conservancy in the genetic code. Thus, some regions of SEQ ID NO: 1 and its complement will require longer segments to be unique while others will require only short segments, typically between 24 and 32 nucleotides or more in length (e.g. 24, 25, 26, 27, 28, 29, 30,31, 32, 33, 34, 35, 36, 37, 38, 39, 40, 41, 42, 43, 44, 45, 46, 47, 48, 49, 50, 51, 52, 53, 54, 55, or more), including every integer up to the entire length of the disclosed sequence less one. Many segments of the polynucleotide coding region or complements thereof that are 18 or more nucleotides in length will be unique. Those skilled in the art are well versed in methods for selecting such sequences, typically on the basis of the ability of the unique fragment to selectively distinguish the sequence of interest from non-NR3B nucleic acid molecules. A comparison of the sequence of the fragment to those on known databases typically is all that is necessary, although in vitro confirmatory hybridization and sequencing analysis may be performed.

A fragment can be a functional fragment. A functional fragment of a nucleic acid molecule of the invention is a fragment which retains some functional property of the larger nucleic acid molecule, such as coding for a functional polypeptide, regulating cation passage in NMDA receptors, regulating ligand binding, regulating conformation of pore, regulating transcription of operably linked nucleic acid molecules, and the like. One of ordinary skill in the art can readily determine using the assays described herein and those well known in the art to determine whether a fragment is a functional fragment of a nucleic acid molecule using no more than routine experimentation.

In yet another aspect of the invention, mutant NR3B nucleic acid molecules are provided, which do not encode fully functional NR3B polypeptides. Rather, these mutant NR3B nucleic acid molecules of the invention contain a sequence which is identical to SEQ ID NO: 1 with the exception that the sequence includes one or more mutations, e.g., deletions, additions or substitutions, such that the mutant NR3B nucleic acid molecules encode mutant NR3B polypeptides, i.e., polypeptides that do not exhibit 100% of NR3B polypeptide functional activity. It is understood that some mutants will encode non-functional NR3B polypeptides, and other mutants will encode NR3B polypeptides with reduced or enhanced function. For example, a mutant NR3B molecule may encode a NR3B polypeptide that has from 0 through 25% of NR3B polypeptide finctional activity, 26% through 50% of NR3B functional activity, 51% through 75% of NR3B polypeptide finctional activity, or 76% through 95% of NR3B polypeptide fuctional activity, as assessed, for example, by the electrophysiological assay for NMDA receptors and NR3B function described in the Examples. It will be understood by one of ordinary skill in the art, that some mutant NR3B nucleic acids may encode polypeptides that have over 100% of NR3B polypeptide finctional activity. For example, a mutant may encode a NR3B polypeptide that has from 101% through 125% of NR3B functional activity, 125% through 150% of NR3B functional activity, or 150% through 200% or more of NR3B functional activity as assessed for example, by the electrophysiological assay described in the Examples. The level of finction of a mutant NR3B polypeptide can be determined and compared to that of NR3B polypeptide using standard assays known to one of ordinary skill in the art. Such assays include, but are not limited to the electrophysiological assays described herein. As used herein, the term “affect the functional activity” means to either inhibit or enhance the functional activity.

As used herein with respect to nucleic acid molecules, in general, the term “isolated” means: (i) amplified in vitro by, for example, PCR; (ii) recombinantly produced by cloning; (iii) purified, as by cleavage and gel separation; or (iv) synthesized by, for example, chemical synthesis. An isolated nucleic acid molecule is one which is readily manipulable by recombinant DNA techniques well known in the art. Thus, a nucleotide sequence contained in a vector in which 5′ and 3′ restriction sites are known or for which PCR primer sequences have been disclosed is considered isolated but a nucleic acid sequence existing in, its native state in its natural host is not. An isolated nucleic acid molecule may be substantially purified, but need not be. For example, a nucleic acid molecule that is isolated within a cloning or expression vector is not pure in that it may comprise only a tiny percentage of the material in the cell in which it resides. Such a nucleic acid molecule is isolated, however, as the term is used herein because it is readily manipulable by standard techniques known to those of ordinary skill in the art. An isolated nucleic acid molecule as used herein is not a naturally occurring chromosome.

As used herein, a “mutant NR3B nucleic acid molecule” refers to a NR3B nucleic acid molecule which includes a mutation (addition, deletion, or substitution) such that the mutant NR3B nucleic acid molecule does not encode a fully functional NR3B polypeptide. Rather, the mutant NR3B nucleic acid molecule encodes a mutant NR3B polypeptide, i.e., a polypeptide which does not exhibit the same functional activity as a NR3B polypeptide. Thus, a “mutant NR3B polypeptide” refers to a gene product of a mutant NR3B nucleic acid molecule. As used herein, the term “aberrant” includes decreased expression (including zero expression) of the natural NR3B molecule (nucleic acid or polypeptide) as compared to its expression in a subject who does not have a NR3B-associated disorder, and increased expression of a mutant NR3B molecule (nucleic acid or polypeptide) as compared to its expression in a subject who does not have a NR3B-associated disorder.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to a transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions. Based upon SEQ ID NO: 1 or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnology 14: 840-844, 1996).

Most preferably, the antisense oligonucleotides comprise a complementary sequence of 20-30 bases. Although oligonucleotides may be chosen which are antisense to any region of the gene or its transcripts, in preferred embodiments the antisense oligonucleotides correspond to N-terminal or 5′ upstream sites such as translation initiation, transcription initiation, or promoter sites. In addition, 3′-untranslated regions may be targeted. Targeting to mRNA splicing sites also has been used in the art but may be less preferred because alternative mRNA splicing of the NR3B transcript occurs. In addition, the antisense is targeted, preferably, to sites in which mRNA secondary structure is not expected (see, e.g., Sainio et al., Cell Mol. Neurobiol. 14(5):439-457, 1994) and at which polypeptides are not expected to bind. The present invention also provides for antisense oligonucleotides which are complementary to genomic DNA and/or cDNA corresponding to SEQ ID NO: 1 or SEQ ID NO: 3. Antisense to allelic or homologous cDNAs and genomic DNAs are enabled without undue experimentation. As mentioned above, the invention embraces antisense oligonucleotides that selectively bind to a mutant NR3B nucleic acid molecule encoding a mutant NR3B polypeptide. This is desirable in medical conditions wherein an aberrant NR3B expression is not desirable.

In one set of embodiments of the aforementioned human and mouse compositions and utilities, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art-recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways, which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus, modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, nucleic acid molecules encoding NR3B polypeptides, together with pharmaceutically acceptable carriers.

Antisense oligonucleotides may be administered as part of a pharmaceutical composition. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

According to yet another aspect of the invention, an expression vector comprising any of the isolated nucleic acid molecules of the invention, preferably operably linked to a promoter is provided. In a related aspect, host cells transformed or transfected with such expression vectors also are provided.

As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding polypeptides which increase or decrease either resistance or sensitivity to antibiotics or other compounds, genes which encode enzymes whose activities are detectable by standard assays known in the art (e.g., β-galactosidase, alkaline phosphatase, or luciferase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional polypeptide, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a polypeptide. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include S′ leader or signal sequences. The choice and design of an appropriate vector is within the ability and discretion of one of ordinary skill in the art.

It will also be recognized that the invention embraces the use of the NR3B cDNA sequences or mutant NR3B cDNA sequences in expression vectors, as well as to transfect host cells and cell lines, be these prokaryotic (e.g., E. coli), or eukaryotic (e.g., CHO cells, COS cells, HEK293 cells, Xenopus oocytes, yeast expression systems and recombinant baculovirus expression in insect cells). Especially useful are mammalian cells such as human, mouse, hamster, pig, goat, primate, etc. They may be of a wide variety of tissue types, including neuronal cells, fibroblasts, oocytes, monocytes, lymphocytes, and they may be primary cells or cell lines. Specific examples include keratinocytes, neuronal cells, and embryonic stem cells. The expression vectors require that the pertinent sequence, i.e., those nucleic acids described herein, be operably linked to a promoter.

In some embodiments, NR3B subunit-encoding DNA is ligated into a vector, and introduced into suitable host cells to produce transformed cell lines that express a specific NMDA receptor subtype, or specific combinations of subunits, including NR3B. The resulting cell lines can then be produced in quantity for reproducible quantitative analysis of the effects of known or potential drugs on receptor function. In other embodiments, mRNA may be produced by in vitro transcription of DNA encoding each subunit. This mRNA, either from a single subunit clone or from a combination of clones, can then be injected into Xenopus oocytes using standard procedures known to those of ordinary skill in the art, where the mRNA directs the synthesis of the receptor subunits, which then form functional receptors. Alternatively, the subunit-encoding DNA can be directly injected into oocytes for expression of functional receptors. The transfected mammalian cells or injected oocytes may then be used in the methods of drug screening provided herein.

Eukaryotic cells in which DNA or RNA may be introduced include any cells that are transfectable by such DNA or RNA or into which such DNA or RNA may be injected. Preferred cells are those that can be transiently or stably transfected and also express the DNA and RNA. Presently most preferred cells are those that can form recombinant or heterologous NMDA receptors comprising one or more subunits encoded by the heterologous DNA. Such cells may be identified empirically or selected from among those known to be readily transfected or injected.

Exemplary cells for introducing DNA include cells of mammalian origin (e.g., COS cells, mouse L cells, Chinese hamster ovary (CHO) cells, human embryonic kidney (HEK) cells (particularly HBK293 cells that can be frozen in liquid nitrogen and then thawed and regrown; for example, those described in U.S. Pat. No. 5,024,939 to Gorman (see, also, Stillman et al. (1985) Mol. Cell. Biol. 5:2051-2060)), African green monkey cells (and other such cells known to those of skill in the art), amphibian cells (e.g., Xenopus laevis oocytes), yeast cells (e.g., Saccharomyces cerevisiae, Pichia pastoris), and the like. Exemplary cells for expressing injected RNA transcripts include Xenopus laevis oocytes. Cells for transfection of DNA are known to those of skill in the art or may be empirically identified, and include HEK293 (which are available from ATCC under accession #CRL 1573); Ltk⁻¹ cells (which are available from ATCC under accession #CCL1.3); COS-7 cells (which are available from ATCC under accession #CRL 1651); and DG44 cells (dhfr CHO cells; see, e.g., Urlaub et al. (1986) Cell. Molec. Genet. 12:555).

DNA may be stably incorporated into cells or may be transiently expressed using methods known in the art. Stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene (such as the E. coli β-galactosidase gene) to monitor transfection efficiency. Selectable marker genes usually are not included in the transient transfections because the transfectants are typically not grown under selective conditions, and are usually analyzed within a few days after transfection.

To produce such stably or transiently transfected cells, the cells should be transfected with a sufficient concentration of subunit-encoding nucleic acids to form NMDA receptors that contain the subunits encoded by heterologous DNA. The precise amounts and ratios of DNA encoding the subunits may be empirically determined and optimized for a particular combination of subunits, cells and assay conditions. Recombinant cells that express NMDA receptors containing subunits encoded only by the heterologous DNA or RNA are especially preferred.

Heterologous DNA may be maintained in the cell as an episomal element or may be integrated into chromosomal DNA of the cell. The resulting recombinant cells may then be cultured or subcultured (or passaged, in the case of mammalian cells) from such a culture or a subculture thereof. Methods for transfection, injection and culturing recombinant cells are known to the skilled artisan.

As used herein, the terms “heterologous” or foreign” DNA and RNA are used interchangeably and refer to DNA or RNA that does not occur naturally as part of the genome of the cell in which it is present or to DNA or RNA that is found in a location or locations in the genome that differ from that in which it occurs in nature. Typically, heterologous or foreign DNA and RNA refers to DNA or RNA that is not endogenous to the host cell and has been artificially introduced into the cell. Examples of heterologous DNA include DNA that encodes a NMDA receptor subunit, DNA that encodes RNA or polypeptides that mediate or alter expression of endogenous DNA by affecting transcription, translation, or other regulatable biochemical processes, and the like. The cell that expresses heterologous DNA may contain DNA encoding the same or different expression products. Heterologous DNA need not be expressed and may be integrated into the host cell genome or maintained episomally.

Recombinant receptors on eukaryotic cell surfaces may contain one or more subunits encoded by the DNA or mRNA encoding NMDA receptor subunits, or may contain a mixture of subunits encoded by the host cell and subunits encoded by heterologous DNA or mRNA. Recombinant receptors may be homomeric or may be a heteromeric combination of multiple subunits. Mixtures of DNA or mRNA encoding receptors subunits from various species, such as mice and humans, may also be introduced into the cells. Thus, a cell can be prepared that expresses recombinant receptors containing only NR3B subunits, or a combination of any one or more NR1, NR2A-NR2D, or NR3A and any one or more NR3B subunits provided herein. For example, NR3B subunits of the present invention can be co-expressed with NR1 and/or NR2A receptor subunits. Specific examples of heteromeric combinations of recombinant human NR3B subunits that have been expressed in HEK293 cells include NR3B+NR1 and NR3B+NR2A (see Examples).

In general, the conditional expression vectors used in such systems use a variety of promoters which confer the desired gene expression pattern (e.g., temporal or spatial). Conditional promoters also can be operably linked to NR3B nucleic acid molecules to increase or decrease expression of a NR3B molecule in a regulated or conditional maimer. Trans-acting negative or positive regulators of NR3B activity or expression also can be operably linked to a conditional promoter as described above. Such trans-acting regulators include antisense NR3B nucleic acid molecules, nucleic acid molecules which encode dominant negative NR3B molecules, ribozyme molecules specific for NR3B nucleic acid molecules, and the like. (see Sze, S. C., et al., Neurochem Int. October 2001;39(4):319-27). The transgenic non-human animals are useful inter alia, for testing biochemical or physiological effects of diagnostics or therapeutics for conditions characterized by altered NR3B molecule expression or function. Other uses will be apparent to one of ordinary skill in the art. Thus, the invention also permits the construction of NR3B gene “knock-outs” in cells and in animals, providing materials for studying certain aspects of NMDA receptor disorders and/or NR3B-associated motor neuron diseases.

The invention also permits the construction of NR3B polypeptide gene “knock-outs” or “knock-ins” in cells and in animals, providing materials for studying certain aspects of NR3B-associated motor neuron diseases and immune system responses to NR3B-associated motor neuron diseases by regulating the expression of NR3B polypeptides. For example, a knock-in mouse may be constructed and examined for clinical parallels between the model and a NR3B-associated motor neuron disease affected mouse with upregulated expression of a normal or mutated NR3B polypeptide. Such a cellular or animal model may be useful for assessing treatment strategies for NR3B -associated motor neuron diseases. An example of a “knock-in” mouse, although not intended to be limiting, involves introducing a NR3B molecule into the cell line and introducing that cell line into a mouse. A “knock-in” model provides a model with which to evaluate the effects of candidate pharmaceutical compounds (e.g. inhibitory effects) on a living animal that expresses a NR3B molecule.

Alternative types of animal models for NR3B-associated motor neuron diseases may be developed based on the invention and may provide a model in which to test treatments, and assess the etiology of NR3B-associated motor neuron diseases.

According to another aspect of the invention, a transgenic non-human animal comprising an expression vector of the invention is provided, including a transgenic non-human animal which has reduced expression of a NR3B nucleic acid molecule or elevated expression of a NR3B or mutant NR3B nucleic acid molecule.

Thus the transgenic animal include “knock-out” animals having a homozygous or heterozygous gene disruption by homologous recombination, animals having episomal or chromosomally incorporated expression vectors, etc. Knock-out animals can be prepared by homologous recombination using embryonic stem cells as is well known in the art. The recombination can be facilitated by the cre/lox system or other recombinase systems known to one of ordinary skill in the art. In certain embodiments, the recombinase system itself is expressed conditionally, for example, in certain tissues or cell types, at certain embryonic or post-embryonic developmental stages, inducibly by the addition of a compound which increases or decreases expression, and the like.

The invention also embraces so-called expression kits, which allow the artisan to prepare a desired expression vector or vectors. Such expression kits include at least separate portions of each of the previously discussed coding sequences. Other components may be added, as desired, as long as the previously mentioned sequences, which are required, are included.

According to another aspect of the invention, an isolated NR3B polypeptide encoded by any of the foregoing isolated nucleic acid molecules of the invention is provided. As used herein, a NR3B polypeptide refers to a polypeptide which is encoded by a nucleic acid having SEQ ID NO: 1, a functional fragment thereof, or a functional equivalent thereof (e.g., a nucleic acid sequence encoding the same polypeptide as encoded by SEQ ID NO: 1), provided that the functional fragment or equivalent encodes a NR3B polypeptide which exhibits a NR3B functional activity. As used herein, a NR3B functional activity refers to the ability of a NR3B polypeptide to modulate one or more of the following parameters: NMDA receptor activity, cation passage in NMDA receptor channel, and/or NMDA receptor ligand binding. An exemplary NR3B functional activity is modulation of NMDA receptor activity and ion flow following ligand binding to a NMDA receptor. NR3B functional activity can be determined by measuring electrophysiological changes in cation passage at the NMDA receptor using, for example, the assays described in the Examples.

Preferably, the isolated polypeptide comprises the amino acid sequence set forth as: SEQ ID NO: 2 or fragments of SEQ ID NO: 2. Such polypeptides are useful, for example, alone or as fusion polypeptides to generate antibodies for NR3B polypeptides. The polypeptides of the invention are also useful in animal models of motor neuron disease, for example, an NR3B polypeptide of the invention may be expressed as a receptor subunit in a NMDA receptor and its expression and/or function may be monitored using methods described herein to determine the function of the NR3B polypeptide NMDA receptor subunit in motor neuron disease. In addition, expression and/or function of the NR3B polypeptide of the invention can be modulated using methods described herein and the effect on the NMDA receptor activity can be evaluated as a method of determining the disease process in motor neuron disease such as ALS. In addition, the multimeric NMDA receptors containing NR3B and other subunits can be generated and expressed in cells, for example cells in culture, using methods described herein. These cells may be useful for examining functional activity variations of different subunit combinations in NMDA receptors, and may also be useful in screening assays for evaluating the effect of candidate pharmacological compounds on function of a NR3B subunit of a glutamate receptor. The animal models including NR3B polypeptide NMDA receptor subunits of the invention are also useful for such screening assays.

The invention also provides functional polypeptide fragments, and functional combinations thereof, encoded by the DNAs of the invention. Such functional polypeptide fragments can be produced by those skilled in the art, without undue experimentation, by eliminating some or all of the amino acids in the sequence not essential for the polypeptide to function as a glutamate receptor subunit. A determination of the amino acids that are essential for glutamate receptor function is made, for example, by systematic digestion of the DNAs encoding the polypeptides and/or by the introduction of deletions into the DNAs. The modified (e.g., deleted or digested) DNAs are expressed, for example, by transcribing the DNA and then introducing the resulting mRNA into Xenopus oocytes, where translation of the mRNAs will occur. Functional analysis of the polypeptides thus expressed in the oocytes is accomplished by exposing the oocytes to ligands known to bind to and functionally activate glutamate receptors, and then monitoring the oocytes to see if the expressed fragments form ion channel(s). If ion channel(s) are detected, the fragments are functional as glutamate receptor subunits. (see U.S. Pat. No. 6,111,091)

Polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed polypeptide. Short polypeptides, including antigenic peptides (such as those presented by MHC molecules on the surface of a cell for immune recognition) also can be synthesized chemically using well-established methods of peptide synthesis.

Thus, as used herein with respect to polypeptides, “isolated” means separated from its native environment and present in sufficient quantity to permit its identification or use. Isolated, when referring to a protein or polypeptide, means, for example: (i) selectively produced by expression of a recombinant nucleic acid or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated polypeptide may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in living systems, e.g. isolated from other polypeptides.

A fragment of a NR3B polypeptide, for example, generally has the features and characteristics of fragments including unique fragments as discussed above in connection with nucleic acid molecules. As will be recognized by those skilled in the art, the size of a fragment which is unique will depend upon factors such as whether the fragment constitutes a portion of a conserved protein domain. Thus, some regions of NR3B polypeptide, for example a conserved binding domain, may require longer segments to be unique. Others will require only short segments, typically between 5 and 14 amino acids (e.g., 5, 6, 7, 8, 9, 10, 11, 12, 13, and 14 amino acids long) as used to generate NR3B-specific antibodies.

Fragments of a polypeptide preferably are those fragments which retain a distinct functional capability of the polypeptide. Functional capabilities which can be retained in a fragment of a polypeptide include interaction with antibodies, interaction with other polypeptides or fragments thereof, selective binding of nucleic acid molecules, and enzymatic activity. Functional activity of the NMDA receptor as a whole can also be assessed with respect to the identity of fragments of NR3B that allow the NMDA receptor to maintain normal function. For example, fragments can be tested in a cell or animal model to determine the size and sequence of fragments that when expressed as a subunit of an NMDA receptor, allow normal or aberrant function of that receptor. One important activity is the ability to act as a signature for identifying the polypeptide. Another is the ability to provoke an immune response to a mutant NR3B molecule but not provoke an immune response to normal levels of a nonmutated NR3B molecule. For example, identification of a fragment of a mutant NR3B subunit that is antigenic, in contrast to a non-antigenic normal NR3B subunit.

Those skilled in the art are well versed in methods for selecting unique amino acid sequences, typically on the basis of the ability of the fragment to selectively distinguish the sequence of interest from non-family members. A comparison of the sequence of the fragment to those on known databases typically is all that is necessary.

The invention embraces variants of the NR3B polypeptides described herein. As used herein, a “variant” of a NR3B polypeptide is a polypeptide which contains one or more modifications to the primary amino acid sequence of a NR3B polypeptide. Modifications which create a NR3B polypeptide variant can be made to a NR3B polypeptide 1) increase, reduce, or eliminate an activity of the NR3B polypeptide; 2) to enhance a property of the NR3B polypeptide, such as polypeptide stability in an expression system or the stability of protein-protein binding; or 3) to provide a novel activity or property to a NR3B polypeptide, such as addition of an antigenic epitope or addition of a detectable moiety.

Modifications to a NR3B polypeptide are typically made to the nucleic acid molecule which encodes the polypeptide, and can include deletions, point mutations, truncations, amino acid substitutions, and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the NR3B amino acid sequences. One of skill in the art will be familiar with methods for predicting the effect on polypeptide conformation of a change in polypeptide sequence, and can thus “design” a variant NR3B polypeptide according to known methods. One example of such a method is described by Dahiyat and Mayo in Science 278:82-87, 1997, whereby polypeptides can be designed de novo. The method can be applied to a known polypeptide to vary only a portion of the polypeptide sequence. By applying the computational methods of Dahiyat and Mayo, specific variants of a NR3B polypeptide can be proposed and tested to determine whether the variant retains a desired conformation.

In general, variants include NR3B polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a NR3B polypeptide by eliminating proteolysis by proteases in an expression system (e.g., dibasic amino acid residues in yeast expression systems in which KEX2 protease activity is present).

Mutations of a nucleic acid molecule which encode a NR3B polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant NR3B polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a NR3B gene or cDNA clone to enhance expression of the polypeptide. The activity of variants of NR3B polypeptides can be tested by cloning the gene encoding the variant NR3B polypeptide into a bacterial, amphibian, or mammalian expression vector, introducing the vector into an appropriate host cell, expressing the variant NR3B polypeptide, and testing for a functional capability of the NR3B polypeptide as disclosed herein. Preparation of other variant polypeptides may favor testing of other activities, as will be known to one of ordinary skill in the art.

The skilled artisan will also realize that conservative amino acid substitutions may be made in NR3B polypeptides to provide functional variants of the foregoing polypeptides, i.e., the variants which the functional capabilities of the NR3B polypeptides. As used herein, a “conservative amino acid substitution” refers to an amino acid substitution which does not alter the relative charge or size characteristics of the polypeptide in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a polypeptide derived from a NR3B polypeptide possesses modulator activity such as suppressing/reducing NMDA receptor function and/or cation passage, one can make conservative amino acid substitutions to the amino acid sequence of the polypeptide. The substituted polypeptides can then be tested for one or more of the above-noted functions, in vivo or in vitro. These variants can be tested for improved stability and are useful, inter alia, in pharmaceutical compositions.

Functional variants of NR3B polypeptides, i.e., variants of polypeptides which retain the function of the NR3B polypeptides, can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functional variants of the NR3B polypeptides include conservative amino acid substitutions of the polypeptides encoded by SEQ ID NO: 2 and SEQ ID NO: 4. Conservative amino-acid substitutions in the amino acid sequence of NR3B polypeptides to produce functional variants of NR3B polypeptides typically are made by alteration of the nucleic acid molecule encoding a NR3B polypeptide (e.g., SEQ ID NO:1). Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a NR3B polypeptide. Where amino acid substitutions are made to a small unique fragment of a NR3B polypeptide, the substitutions can be made by directly synthesizing the polypeptide. The activity of functional variants or fragments of NR3B polypeptide can be tested by cloning the gene or transcript that encodes the altered NR3B polypeptide into a bacterial, mammalian, or insect cell expression vector, introducing the vector into an appropriate host cell, expressing the altered NR3B polypeptide, (or by expressing the altered NR3B in oocytes as described herein) and testing for a functional capability of the NR3B polypeptides as disclosed herein.

The invention as described herein has a number of uses, some of which are described elsewhere herein. First, the invention permits isolation of the NR3B polypeptide molecules. A variety of methodologies well known to the skilled practitioner can be utilized to obtain isolated NR3B molecules. The polypeptide may be purified from cells which naturally produce the polypeptide by chromatographic means or immunological recognition. Alternatively, an expression vector may be introduced into cells to cause production of the polypeptide. In another method, mRNA transcripts may be microinjected or otherwise introduced into cells to cause production of the encoded polypeptide, as described herein. Translation of mRNA in cell-free extracts such as the reticulocyte lysate system also may be used to produce polypeptide. Those skilled in the art also can readily follow known methods for isolating NR3B polypeptides. These include, but are not limited to, HPLC, size-exclusion chromatography, ion-exchange chromatography, and immune-affinity chromatography. In addition, those skilled in the art will also be able to utilize recombinant NR3B to determine the structure (e.g. quaternary, tertiary) of the NR3B polypeptide or its variants.

The isolation and identification of NR3B nucleic acid molecules also makes it possible for the artisan to diagnose a disorder characterized by aberrant expression or function of a NR3B nucleic acid molecule or polypeptide. These methods involve determining the aberrant expression of one or more NR3B nucleic acid molecules and/or encoded NR3B polypeptides. In the former situations, such determinations can be carried out via any standard nucleic acid determination assay, including the PCR, or assaying with labeled hybridization probes. In the latter situations, such determinations can be carried out by screening patient antisera for recognition of the polypeptide or by assaying biological samples with binding partners (e.g., antibodies) for NR3B polypeptides.

The invention also provides, in certain embodiments, “dominant negative” polypeptides derived from NR3B polypeptides. A dominant negative polypeptide is an inactive variant of a polypeptide, which, by interacting with the cellular machinery, displaces an active polypeptide from its interaction with the cellular machinery or competes with the active polypeptide, thereby reducing the effect of the active polypeptide. Dominant negative polypeptides are useful, or example, for preparing transgenic non-human animals to further characterize the functions of the NR3B molecules disclosed herein. For example, a dominant negative receptor which binds a ligand but does not transmit a signal in response to binding of the ligand can reduce the biological effect of expression of the ligand. Similarly, a dominant negative transcription factor which binds to a promoter site in the control region of a gene but does not increase gene transcription can reduce the effect of a normal transcription factor by occupying promoter binding sites without increasing transcription. Another example of a dominant negative NR3B is a mutant form of this polypeptide lacking a ligand-binding domain.

The end result of the expression of a dominant negative polypeptide in a cell is an altered function of active polypeptides. One of ordinary skill in the art can assess the potential for a dominant negative variant of a polypeptide, and using standard mutagenesis techniques to create one or more dominant negative variant polypeptides. For example, one of ordinary skill in the art can modify the sequence of NR3B polypeptides by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized polypeptides for diminution in a selected and/or for retention of such an activity. Other similar methods for creating and testing dominant negative variants of a polypeptide will be apparent to one of ordinary skill in the art.

In yet a further aspect of the invention, binding polypeptides that selectively bind to a NR3B molecule are provided. According to this aspect, the binding polypeptides bind to an isolated nucleic acid or polypeptide of the invention, including binding to unique fragments thereof. Preferably, the binding polypeptides bind to a NR3B polypeptide or a fragment thereof. In certain particularly preferred embodiments, the binding polypeptide binds to a mutant NR3B polypeptide but does not bind to a non-mutant NR3B polypeptide, i.e., the binding polypeptides are selective for binding to the mutant NR3B polypeptide and can be used in various assays to detect the presence of the mutant NR3B polypeptide without detecting non-mutant NR3B polypeptide. Such mutant N3B polypeptide binding polypeptides also can be used to selectively bind to a mutant NR3B molecule in a cell (in vivo or ex vivo) for imaging and therapeutic applications in which, for example, the binding polypeptide is tagged with a detectable label and/or a toxin for targeted delivery to the mutant NR3B molecule. In other preferred embodiments, the binding polypeptide binds to a NR3B polypeptide but does not bind to a mutant NR3B polypeptide, i.e., the binding polypeptides are selective for binding to the NR3B polypeptide and can be used in various assays to detect the presence of the NR3B polypeptide without detecting mutant NR3B polypeptide.

In preferred embodiments, the binding polypeptide is an antibody or antibody fragment, such as an Fab or F(ab)₂ fragment of an antibody. Typically, the fragment includes a CDR3 region that is selective for the NR3B polypeptide. Any of the various types of antibodies can be used for this purpose, including monoclonal antibodies, humanized antibodies, and chimeric antibodies.

Thus, the invention provides agents which bind to NR3B polypeptides encoded by NR3B nucleic acid molecules, respectively, and in certain embodiments preferably to unique fragments of the NR3B polypeptides. Such binding partners can be used in screening assays to detect the presence or absence of a NR3B polypeptide and in purification protocols to isolate such NR3B polypeptides. Likewise, such binding partners can be used to selectively target drugs, toxins or other molecules to cells which express mutant NR3B polypeptides. In this manner, cells present in tissues that express mutant NR3B polypeptides can be treated with cytotoxic compounds. Such agents also can be used to inhibit the native activity of the NR3B polypeptides, for example, by binding to such polypeptides, to further characterize the functions of these molecules.

In addition, antibodies generated against the above-described NR3B and mutant NR3B polypeptides are provided. Such antibodies can be employed for studying receptor tissue localization, subunit composition, structure of functional domains, as well as in diagnostic applications, therapeutic applications, and the like

Factors to consider in selecting portions of the NR3B subunit for use as an immunogen (as either a synthetic peptide or a recombinantly produced bacterial fusion protein) include antigenicity, accessibility (i.e., extracellular and cytoplasmic domains), uniqueness to the particular subunit, etc.

The availability of subunit-specific antibodies makes possible the application of the technique of immunohistochemistry to monitor the distribution and expression density of NR3B (e.g., in normal versus diseased brain tissue). Such antibodies could also be employed for diagnostic and therapeutic applications. For example, invention provides methods for modulating the ion channel activity of receptor(s) of the invention by contacting NR3B subunit(s) with an effective amount of the above-described antibodies.

The invention, therefore, provides antibodies or fragments of antibodies having the ability to selectively bind to NR3B polypeptides, and preferably to unique fragments thereof. Antibodies include polyclonal, monoclonal, and chimeric antibodies, prepared, e.g., according to conventional methodology.

The antibodies of the present invention are prepared by any of a variety of methods, including administering polypeptide, fragments of polypeptide, cells expressing the polypeptide or fragments thereof and the like to an animal to induce polyclonal antibodies. Monoclonal antibodies are produced according to techniques well known in the art. As detailed herein, such antibodies may be used for example to identify tissues expressing polypeptide or to purify polypeptide.

Significantly, as is well known in the art, only a small portion of an antibody molecule, the paratope, is involved in the binding of the antibody to its epitope (see, in general, Clark, W. R. (1986) The Experimental Foundations of Modern Immunonology Wiley & Sons, Inc., New York; Roitt, I. (1991) Essential Immunology 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. (see, e.g., U.S. Pat. Nos. 4,816,567, 5,225,539, 5,585,089, 5,693,762, and 5,859,205). Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Fully human monoclonal antibodies also can be prepared by immunizing mice transgenic for large portions of human immunoglobulin heavy and light chain loci. -Following immunization of-these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies. An anti-peptide approach is another methodology that can be used to generate new antibodies.

Thus, the invention involves polypeptides of numerous size and type that bind specifically to NR3B polypeptides. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent a completely degenerate or biased array. One then can select phage-bearing inserts which bind to a NR3B polypeptide. This process can be repeated through several cycles of reselection of phage that bind to a NR3B polypeptide. Repeated rounds lead to enrichment of phage bearing particular sequences. DNA sequence analysis can be conducted to identify the sequences of the expressed polypeptides. The minimal linear portion of the sequence that binds to the NR3B polypeptide can be determined. One can repeat the procedure using a biased library containing inserts containing part or all of the minimal linear portion plus one or more additional degenerate residues upstream or downstream thereof. Thus, the NR3B polypeptides of the invention can be used to screen peptide libraries, including phage display libraries, to identify and select peptide binding partners of the NR3B polypeptides of the invention. Such molecules can be used, as described, for screening assays, for diagnostic assays, for purification protocols or for targeting drugs, toxins and/or labeling agents (e.g. radioisotopes, fluorescent molecules, etc). to cells which express mutant NR3B genes such as neuronal cells which have aberrant NR3B expression.

As detailed herein, the foregoing antibodies and other binding molecules may be used for example to identify tissues expressing NR3B polypeptide or to purify NR3B polypeptide. Antibodies also may be coupled to specific diagnostic labeling agents for imaging NR3B expression in cells and tissues, and coupled to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99m, iodine-131 and indium-111, nuclides for nuclear magnetic resonance such as fluorine and gadolinium. Other diagnostic agents useful in the invention will be apparent to one of ordinary skill in the art.

In some circumstances, it may be preferred to conjugate molecules to a compound which facilitates transport of the molecule across the blood-brain barrier (BBB) for transport into the central nervous system. Such molecules for transport may include, but are not limited to: NR3B, NR3B-binding polypeptide (for example an anti-NR3B antibody), NMDA receptor agonist, NMDA receptor antagonist, and the like. As used herein, a compound which facilitates transport across the BBB is one which, when conjugated to the molecule to be transported, facilitates the amount of that molecule delivered to the brain as compared with non-conjugated molecule. The compound can induce transport across the BBB by any mechanism, including receptor-mediated transport, and diffusion. The molecule to be transported can be conjugated to such compounds by well-known methods, including bifunctional linkers, formation of a fusion polypeptide, and formation of biotin/streptavidin or biotin/avidin complexes by attaching either biotin or streptavidin/avidin to the peptide and the complementary molecule to the BBB-transport facilitating compound.

Compounds which facilitate transport across the BBB include transferring receptor binding antibodies (U.S. Pat. No. 5,527,527); certain lipoidal forms of dihydropyridine (see, e.g., U.S. Pat. No. 5,525,727); carrier peptides, such as cationized albumin or Metenkephalin (and others disclosed in U.S. Pat. Nos. 5,442,043; 4,902,505; and 4,801,575); cationized antibodies (U.S. Pat. No. 5,004,697); and fatty acids such as docosahexanoic acid (DHA; U.S. Pat. No. 4,933,324).

For other uses of the molecules to be transported, it may be preferred to administer the molecules in combination with a compound which increases transport of compounds across the blood-brain barrier (BBB). Such compounds, which need not be conjugated to a molecule for transport, increase the transport of the molecule across the BBB into the brain. A compound which increases transport across the BBB is one, for example, which increases the permeability of the BBB, preferably transiently. Coadministration of a molecule for transport with such a compound permits the molecule to cross a permeabilized BBB. Examples of such compounds include bradykinin and agonist derivatives (U.S. Pat. No. 5,112,596); and receptor-mediated permeabilizers such as A-7 (U.S. Pat. No. 5,268,164 and 5,506,206).

According to a further aspect of the invention, pharmaceutical compositions containing the nucleic acid molecules, polypeptides, and binding polypeptides of the invention are provided. The pharmaceutical compositions contain any of the foregoing therapeutic agents in a pharmaceutically acceptable carrier. Thus, in a related aspect, the invention provides a method for forming a medicament that involves placing a therapeutically effective amount of the therapeutic agent in the pharmaceutically acceptable carrier to form one or more doses.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712). Those of skill in the art can readily determine the various parameters and conditions for producing antibody aerosols without resort to undue experimentation. When using antisense preparations of the invention, slow intravenous administration is preferred. Preparations for administering therapeutics may also include compounds for transport across the blood-brain barrier as described elsewhere herein.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The preparations of the invention are administered in effective amounts. An effective amount is that amount of a pharmaceutical preparation that alone, or together with further doses, stimulates the desired response. In the case of treating a NR3B-associated motor neuron disorder, the desired response is inhibiting the progression of the NR3B -associated motor neuron disorder. This may involve only slowing the progression of the disease temporarily, although more preferably, it involves halting the progression of the disease permanently. In the case of stimulating an immune response, e.g. against a mutant NR3B polypeptide, the desired response is an increase in antibodies or T lymphocytes which are specific for the immunogen(s) employed. These responses can be monitored by routine methods or can be monitored according to diagnostic methods of the invention discussed herein.

The therapeutically effective amount of the NR3B molecule is that amount effective to modulate NR3B functional activity levels and reduce, prevent, delay onset, or eliminate the NR3B-associated motor neuron disorder. For example, using the assays described in the Example section, testing can be performed to determine the NR3B functional activity in a subject's tissue and/or cells. Additional tests useful for monitoring the onset, progression, and/or remission, of NR3B-associated motor neuron disorders such as ALS, and other NR3B-associated motor neuron disorders known to one of ordinary skill in the art. As would be understood by one of ordinary skill, for some motor neuron disorders (e.g. reduced NR3B-function disorders) an effective amount would be the amount of NR3B molecules that increases NR3B functional activity to a level that diminishes the disorder, as determined by the aforementioned tests. It is also understood that in other motor neuron disorders (e.g. increased function disorders) an effective amount would be that amount of NR3B molecules that decreases NR3B functional activity to a level that diminishes the disorder, as determined by the aforementioned tests.

The NR3B molecule dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

According to another aspect of the invention, various diagnostic methods are provided. In general, the methods are for diagnosing a motor neuron disorder characterized by aberrant expression of a NR3B molecule. As used herein, “aberrant expression” is dependent upon the particular motor neuron disorder (i.e., characterized by increased or decreased NR3B molecule expression). Thus “aberrant expression refers to increased expression of normal NR3B, decreased expression (including no expression) of a NR3B molecule (nucleic acid or polypeptide), or increased expression of a “mutant NR3B molecule”. A mutant NR3B molecule refers to a NR3B nucleic acid molecule which includes a mutation (deletion, addition, or substitution) or to a NR3B polypeptide molecule (e.g., gene product of mutant NR3B nucleic acid molecule) which includes a mutation, provided that the mutation results in a mutant NR3B polypeptide that has reduced or no non-mutant NR3B polypeptide functional activity. The diagnostic methods of the invention can be used to detect the presence of a disorder associated with aberrant expression of a NR3B molecule, as well as to assess the progression and/or regression of the disorder such as in response to treatment (e.g., chemical therapy).

Expression of NR3B can be evaluated using standard methods known to those of ordinary skill in the art. Such methods include, but are not limited to: PCR, RT-PCT and antibody methods, which can be used to evaluate changes/alternations (for example, relative to normal) in the expression of NR3B at the gene, transcript, and polypeptide levels, respectively. The above-mentioned methods can be used to evaluate expression of NR3B molecule, whether or not it has normal functional activity. For example, a NR3B polypeptide maybe expressed but lack normal function due to mutation that does not effect expression. The above-mentioned methods can be used to evaluate NR3B expression regardless of functional activity.

Expression of NR3B can also be evaluated by determining the functional activity of NR3B in cells and tissues with methods including, but not limited to, the electrophysiological activity assay described in the Examples. According to this aspect of the invention, the method for diagnosing a disorder characterized by aberrant expression of a NR3B molecule involves: detecting in a first biological sample obtained from a subject, expression of a NR3B molecule, wherein decreased or increased expression of a NR3B molecule (depending upon the disorder as discussed herein) compared to a control sample indicates that the subject has a motor neuron disorder characterized by aberrant expression of a NR3B molecule.

As used herein, a “motor neuron disorder characterized by aberrant expression of a NR3B molecule” refers to a motor neuron disorder in which there is a detectable difference in the expression and/or function levels of NR3B molecule(s) in selected cells of a subject compared to the control levels of these molecules. Thus, a disorder characterized by aberrant expression of a NR3B molecule embraces overexpression of NR3B, underexpression (including no expression) of a NR3B molecule compared to control levels of these molecules, as well as expression of a mutant NR3B nucleic acid molecule or mutant NR3B polypeptide. Such differences in expression and/or function levels can be determined in accordance with the diagnostic methods of the invention as disclosed herein. Disorders that are characterized by aberrant expression of a NR3B molecule include: motor neuron disorders associated with abnormal NMDA receptor function, and/or receptor-ligand interaction. Exemplary NR3B-associated motor neuron disorders include, but are not limited to ALS.

In other motor neuron diseases and disorders, an increase in NR3B expression levels and/or functional activity would indicate the presence of an “increased NR3B-activity disorder” in the subject. In addition, the expression of a mutant NR3B that has NR3B-like functional activity, can also indicate the presence of a motor neuron disorder, characterized by an increase in NR3B expression (e.g. functional activity), as described herein. Thus, depending upon the nature of the disorder, i.e., whether it is attributable to reduced or elevated NR3B molecule expression, one skilled in the art can select the appropriate type of treatment to modulate NR3B expression to achieve levels of these molecules which are found in individuals who are not diagnosed with such motor neuron disorders.

In certain embodiments, the methods of the invention are useful to diagnose a NR3B-associated motor neuron disorders including, but not limited to ALS.

In yet other embodiments, the diagnostic methods are useful for diagnosing the progression of a motor neuron disorder and evaluating its treatment. According to these embodiments, the methods further involve: detecting in a second biological sample obtained from the subject, expression of a NR3B molecule, and comparing the expression of the NR3B molecule in the first biological sample and the second biological sample. In these embodiments, a decrease or an increase in the expression of the NR3B molecule in the second biological sample compared to the first biological sample indicates progression of the disorder.

In yet other embodiments, the diagnostic methods are useful for diagnosing the regression of a motor neuron disorder. According to these embodiments, the methods further involve: detecting in a second biological sample obtained from the subject, expression of a NR3B molecule, and comparing the expression of the NR3B molecule in the first biological sample and the second biological sample. In these embodiments, an increase or a decrease in the expression of the NR3B molecule in the second biological sample compared to the first biological sample indicates regression of the disorder. As noted above, it is to be understood by one of ordinary skill that some disorders will be characterized by an increase in NR3B functional activity and other disorders will be characterized by a decrease in NR3B functional activity.

In certain embodiments, the diagnostic methods of the invention detect a NR3B molecule that is a NR3B nucleic acid molecule as described above. In yet other embodiments, the methods involve detecting a NR3B polypeptide as described above.

Various detection methods can be used to practice the-diagnostic methods of the invention. For example, the methods can involve contacting a biological sample with an agent that selectively binds to NR3B molecules to detect these molecules. In certain embodiments, the NR3B molecule is a nucleic acid and the method involves using an agent that selectively binds to the NR3B molecule, e.g., a nucleic acid that hybridizes to SEQ ID NO: 1 under high stringency conditions. In yet other embodiments, the NR3B molecule is a polypeptide and the method involves using an agent that selectively binds to the NR3B molecule, e.g., a binding polypeptide, such as an antibody, that selectively binds to SEQ ID NO: 2.

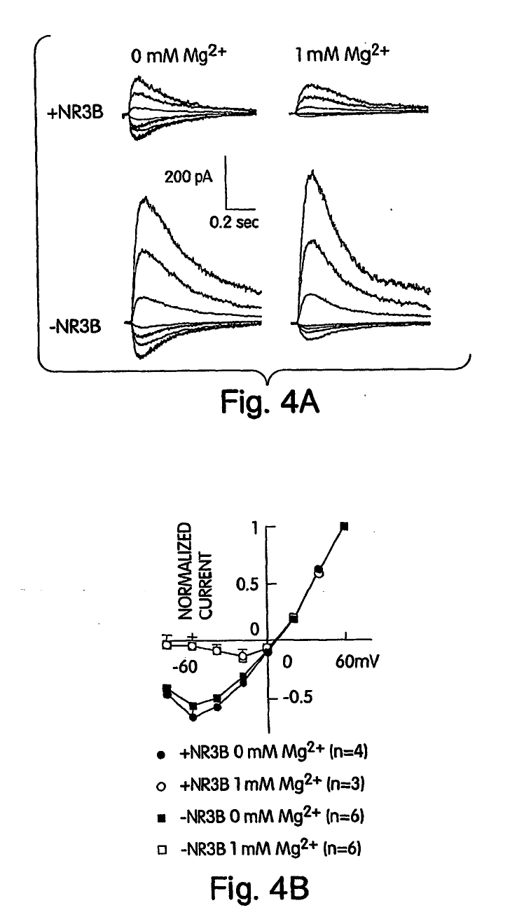

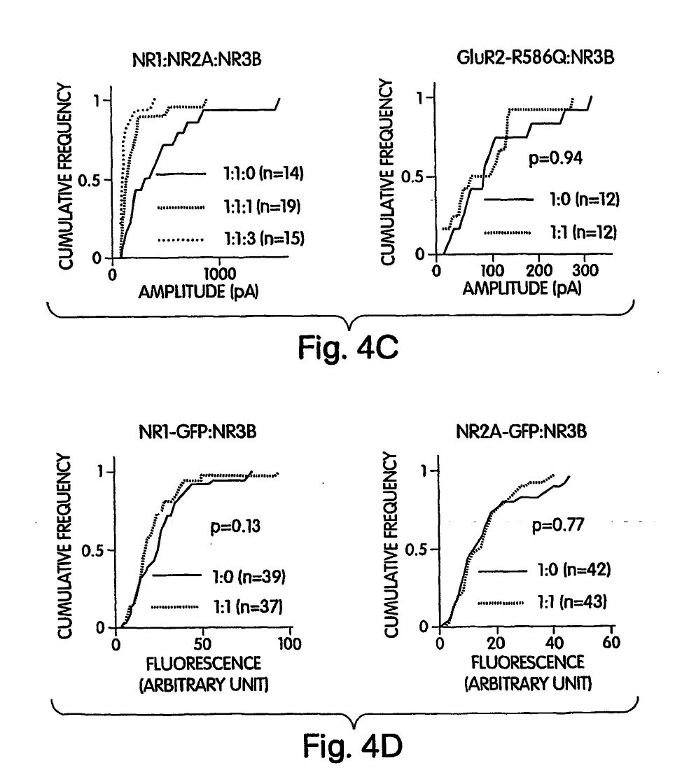

According to still another aspect of the invention, kits for performing the diagnostic methods of the invention are provided. The kits include nucleic acid-based kits or polypeptide-based kits (see FIG. 4). According to the former embodiment, the kits include: one or more nucleic acid molecules that hybridize to a NR3B nucleic acid molecule under high stringency conditions; and instructions for the use of the nucleic acid molecules in the diagnosis of a motor neuron disorder associated with aberrant expression of a NR3B molecule. Nucleic acid-based kits optionally further include a first primer and a second primer, wherein the first primer and the second primer are constructed and arranged to selectively amplify at least a portion of an isolated NR3B nucleic acid molecule comprising SEQ ID NO: 1. Alternatively, polypeptide based-kits are provided. Such kits include: one or more binding polypeptides that selectively bind to a NR3B polypeptide and instructions for the use of the binding polypeptides in the diagnosis of a disorder associated with aberrant expression of a NR3B molecule. In the preferred embodiments, the binding polypeptides are antibodies or antigen-binding fragments thereof, such as those described above. In these and other embodiments, certain of the binding polypeptides bind to the mutant NR3B polypeptide but do not bind to the non-mutant NR3B polypeptide to further distinguish the expression of these polypeptides in a biological sample.

The foregoing kits can include instructions or other printed material on how to use the various components of the kits for diagnostic purposes.

The biological sample can be located in vivo or in vitro. For example, the biological sample can be a tissue in vivo and the agent specific for the NR3B nucleic acid molecule or polypeptide can be used to detect the presence of such molecules in the tissue (e.g., for imaging portions of the tissue that express the NR3B gene products). Alternatively, the biological sample can be located in vitro (e.g., a blood sample, biopsy (e.g., tumor or tissue biopsy), tissue extract). In a particularly preferred embodiment, the biological sample can be a cell-containing sample, more preferably a sample containing neuronal cells. Samples of tissue and/or cells for use in the various methods described herein can be obtained through standard methods. Samples can be surgical samples of any type of tissue or body fluid. Samples can be used directly or processed to facilitate analysis (e.g., paraffin embedding). Exemplary samples include a cell, a cell scraping, a cell extract, a blood sample, spinal fluid sample, a tissue biopsy, including punch biopsy, a tumor biopsy, a bodily fluid, a tissue, or a tissue extract. Samples also can be cultured receptors, cells, tissues, or organs.

NMDA receptors play beneficial roles in excitatory neurotransmission and their ligand-induced activation results in cation passage through the channel. In some motor neuron disorders, such as ALS it may be desirable to increase NR3B expression and/or functional activity, such as by administering a drug that increases NR3B activity in cells and tissues of a subject. Such an increase in activity can be brought about by, for example, increasing expression of NR3B, and/or increasing the level of a mutant NR3B that has an increased level of activity as compared to the activity level of normal NR3B.

In other motor neuron diseases and disorders, it may be desirable to reduce the activity of NR3B to increase cation passage through the NMDA receptor channel. Cation passage maybe increased using methods such as: reducing expression of NR3B and/or inhibiting NR3B activity (e.g. competitive inhibition, binding inhibition). Increasing levels of a mutant NR3B that is non-functional or reduced-function as compared to normal NR3B, can also be therapeutically desirable in diseases and disorders where a reduction in normal NR3B activity is desirable.

In general, the treatment methods involve administering an agent to modulate expression of a NR3B molecule. In certain embodiments, the method for treating a subject with a motor neuron disorder characterized by aberrant expression of a NR3B molecule, involves administering to the subject an effective amount of a NR3B nucleic acid molecule to treat the disorder. In yet other embodiments, the method for treatment involves administering to the subject an effective amount of an antisense molecule to modulate expression of a NR3B nucleic acid molecule and thereby, treat the motor neuron disorder. An exemplary molecule for modulating expression of a mutant NR3B nucleic acid molecule is an antisense molecule that is selective for the mutant nucleic acid and that does not modulate expression of the non-mutant NR3B nucleic acid molecule.

Alternatively, the method for treating a subject with a disorder characterized by aberrant expression of a NR3B molecule involves administering to the subject an effective amount of a NR3B polypeptide to treat the disorder. In some embodiments, the treatment method involves administering to the subject who has a disorder characterized by hypoactivity of NMDA receptors, an effective amount of a binding polypeptide to inhibit a NR3B polypeptide, thereby increase activity of the NMDA receptor and treat the disorder. In other embodiments, the treatment methods involve administering to the subject who has a disorder characterized by hyperactivity of NMDA receptors, an effective amount of a binding polypeptide to increase NR3B activity, thereby decreasing activity of the NMDA receptor. In certain preferred embodiments, the binding polypeptide is an antibody or an antigen-binding fragment thereof; more preferably, the antibodies or antigen-binding fragments are labeled with one or more cytotoxic agents

Some of the foregoing methods of the invention contemplate gene therapy. A procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell(s) of a subject which contains a defective copy of the gene, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art. In vivo gene therapy using vectors such as adenovirus, retroviruses, herpes virus, and targeted liposomes also is contemplated according to the invention.

In preferred embodiments, a virus vector for delivering a nucleic acid molecule encoding a NR3B polypeptide is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus-derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J Immunol 26:1951-1959, 1996). In preferred embodiments, the virus vector is an adenovirus.

Another preferred virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired polypeptides, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Cliffton, N.J. (1991).

Preferably the foregoing nucleic acid delivery vectors: (1) contain exogenous genetic material that can be transcribed and translated in a mammalian cell and that can suppress NR3B-associated disease (e.g. motor neuron-associated disorders), and preferably (2) contain on a surface a ligand that selectively binds to a receptor on the surface of a target cell, such as a mammalian cell, and thereby gains entry to the target cell.

Various techniques may be employed for introducing nucleic acid molecules of the invention into cells, depending on whether the nucleic acid molecules are introduced in vitro or in vivo in a host. Such techniques include transfection of nucleic acid molecule-CaPO₄ precipitates, transfection of nucleic acid molecules associated with DEAE, transfection or infection with the foregoing viruses including the nucleic acid molecule of interest, liposome-mediated transfection, and the like. For certain uses, it is preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane polypeptide on the target cell or a ligand for a receptor on the target cell can be bound to or incorporated within the nucleic acid molecule delivery vehicle. Especially preferred are monoclonal antibodies. Where liposomes are employed to deliver the nucleic acid molecules of the invention, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

In addition to delivery through the use of vectors, NR3B nucleic acids may be delivered to cells without vectors, e.g. as “naked” nucleic acid delivery using methods known to those of skill in the art.

Various forms of the NR3B polypeptide or nucleic acid, as described herein, can be administered and delivered to a mammalian cell (e.g., by virus or liposomes, or by any other suitable methods known in the art or later developed). The method of delivery can be modified to target certain cells, and in particular, cell surface receptor molecules or antigens present on neuronal cells. Methods of targeting cells to deliver nucleic acid constructs are known in the art. The NR3B polypeptide can also be delivered into cells by expressing a recombinant protein fused with peptide carrier molecules, examples of which, though not intended to be limiting, are tat or antennapedia. These delivery methods are known to those of skill in the art and are described in U.S. Pat. No. 6,080,724, and U.S. Pat. No. 5,783,662, the entire contents of which are hereby incorporated by reference.

In addition to the methods described herein for delivering exogenous NR3B, expression of endogenous normal or mutant NR3B can be induced (e.g., upregulated) by the administration of chemicals or other molecules that specifically increase the level of NR3B mRNA and/or polypeptide. Such induction and/or upregulation of endogenous NR3B may occur through methods that include, but not limited to: (a) activation of the NR3B promoter, (b) stabilization of NR3B mRNA, (c) increased translation of NR3B polypeptide and (d) stabilization of NR3B polypeptide.

The invention further provides efficient methods of identifying pharmacological agents or lead compounds for agents which mimic the functional activity of a NR3B molecule. Such NR3B functional activities include excitatory neurotransmission and modulation of cation passage. Generally, the screening methods involve assaying for compounds which modulate (up- or down-regulate) a NR3B functional activity.

The DNA, mRNA, vectors, receptor subunits, receptor subunit combinations and cells provided herein permit production of selected NMDA receptor subunits and specific combinations thereof, as well as antibodies to said receptor subunits. This provides a means to prepare synthetic or recombinant receptors and receptor subunits that are substantially free of contamination from many other receptor polypeptides whose presence can interfere with analysis of a single NMDA receptor subtype. The availability of desired receptor subtypes makes it possible to observe the effect of a drug substance on a particular receptor subtype or combination of NMDA receptor subunits, and to thereby perform initial in vitro screening of the drug substance in a test system that is specific for humans and specific for a human NMDA receptor subtype or combination of NMDA receptor subunits, including NR3B subunits. The availability of specific antibodies makes it possible to identify the subunit combinations expressed in vivo. Such specific combinations can then be employed as preferred targets in drug screening.

The ability to screen drug substances in vitro to determine the effect of the drug on specific receptor compositions should permit the development and screening of receptor subtype-specific or disease-specific drugs. Also, testing of single receptor subunits or specific combinations of various types of receptor subunits with a variety of potential agonists or antagonists provides additional information with respect to the function and activity of the individual subunits and should lead to the identification and design of compounds that are capable of specific interaction with one or more types of receptor subunits (e.g. NR3B) or receptor subtypes. The resulting drugs should exhibit fewer unwanted side effects than drugs identified by screening with cells that express a variety of receptor subtypes.

The invention also provides methods for identifying compounds that bind to human N-methyl-D-aspartate (NMDA) receptor subunit(s) (such as NR3B). For example, NR3B polypeptides can be used in a competitive binding assay. Such an assay can accommodate the rapid screening of a large number of compounds to determine which compounds, if any, are capable of binding to NMDA receptors. Subsequently, more detailed assays can be carried out with those compounds found to bind, to further determine whether such compounds act as modulators, agonists, or antagonists of invention receptors.

Another application of the binding assay of the invention is the assay of test samples (e.g., biological fluids) for the presence or absence of receptors of the present invention. Thus, for example, spinal fluid from a patient displaying symptoms related to glutamatergic pathway dysfunction can be assayed to determine if the observed symptoms are perhaps caused by over- or under-production of receptor subunits, such as NR3B.

The binding assays contemplated by the present invention can be carried out in a variety of ways, as can readily be identified by those of skill in the art. For example, competitive binding assays can be employed, such as radioreceptor assays, fluorescence assays, and the like.

In accordance with a further embodiment of the present invention, there is provided a bioassay for identifying compounds which modulate the activity of NMDA receptors of the invention. An example of such a bioassay includes: exposing cells containing DNA encoding NMDA receptor subunit(s), wherein the cells express functional NMDA receptors, to one or more compounds whose ability to modulate the ion channel activity of the receptors is sought to be determined; and monitoring the cells for changes in ion channel activity.

The above-described bioassay can be used to identify agonists and antagonists for human NMDA receptors. According to this method, recombinant NMDA receptors are contacted with an “unknown” or test substance (in the further presence of a known NMDA agonist, when antagonist activity is being tested), the ion channel activity of the known glutamate receptor is monitored subsequent to the contact with the “unknown” or test substance, and those substances which increase or decrease the ion channel response of the known glutamate receptor(s) are identified as functional ligands (i.e., modulators, agonists or antagonists) for human NMDA receptors. (See electrophysiological assay in Example Section).

In accordance with a particular embodiment of the present invention, recombinant NMDA receptor NR3B-expressing mammalian cells or oocytes can be contacted with a test compound, and the modulating effect(s) thereof can then be evaluated by comparing the NMDA receptor-mediated response in the presence and absence of test compound, or by comparing the response of test cells, or control cells (i.e., cells that do not express NMDA receptor NR3B subunits, or express a normal level of functional NR3B subunits), to the presence of the compound. A recombinant NMDA receptor of the invention includes NR3B subunits and its response to the test compounds may be compared to responses of cells with NMDA receptors lacking NR3B subunits, or containing mutant NR3B subunits.

In accordance with yet another embodiment of the present invention, the ion channel activity of N-methyl-D-aspartate (NMDA) receptors containing NR3B can be modulated by contacting such receptors with an effective amount of at least one compound identified by the above-described bioassay.

A wide variety of assays for pharmacological agents can be used in accordance with this aspect of the invention, including, labeled in vitro protein-protein binding assays, electrophoretic mobility shift assays, immunoassays, cell-based assays such as two- or three-hybrid screens, expression assays, etc. The assay mixture comprises a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with polypeptides and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

An exemplary binding assay is described herein. In general the mixture of the foregoing assay materials is incubated under conditions whereby, but for the presence of the candidate pharmacological agent, the NR3B molecule specifically binds the binding agent (e.g., ligand). The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of specific binding between the NR3B molecule and one or more binding agents is detected by any convenient method available to the user. For cell-free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microfiter plate well, rinsing a bead, particle, chromotograpic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way for cell-based assays such as two- or three-hybrid screens. For cell-free binding assays, one of the components usually comprises, or is coupled to, a detectable label. A wide variety of labels can be used, such as those that provide direct detection (e.g., radioactivity, luminescence, optical or electron density, etc.) or indirect detection (e.g., epitope tag such as the FLAG epitope, enzyme tag such as horseseradish peroxidase, etc.). The label may be bound to a NR3B binding partner (e.g., polypeptide), or incorporated into the structure of the binding partner.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, strepavidin-biotin conjugates, etc. Methods for detecting the labels are well known in the art.

Detection may be effected in any convenient way for cell-based assays such as a calcium influx assay. The calcium influx resulting from activation of the NMDA receptor by ligand binding typically alters a directly or indirectly detectable product, e.g., a calcium sensitive molecule such as fura-2-AM. For cell free binding assays, one of the components usually comprises, or is coupled to, a detectable label. A wide variety of labels can be used, such as those that provide direct detection (e.g., radioactivity, luminescence, optical or electron density, etc). or indirect detection (e.g., epitope tag such as the FLAG epitope, enzyme tag such as horseradish peroxidase, etc.). The label may be bound to a NR3B polypeptide, decoy peptide, or the candidate pharmacological agent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

Thus the present invention includes automated drug screening assays for identifying compositions having the ability to inhibit ion influx in a cell induced by ligand binding, thus contributing to a detectable change in the cytoplasmic level of a predetermined ion in the cell, the cytoplasm of which cell contains an indicator which is sensitive to the ion. The method is carried out in an apparatus which is capable of delivering a reagent solution to a plurality of predetermined cell-containing compartments of a vessel and measuring the detectable change in the cytoplasmic level of the ion in the cells of the predetermined compartments, such as the apparatus and method described in U.S. Pat. No. 6,057,114. Exemplary methods include the following steps. First, a divided culture vessel is provided that has one or more compartments which contain viable cells which, when exposed to NMDA agonists, have a detectable change in the concentration of the predetermined ion in the cytoplasm. The cytoplasms of the cells include an amount of an ion-sensitive fluorescent indicator sufficient to detect a change, if any, in the concentration of the predetermined ion. NMDA agonists are added to the cells to induce calcium influx and/or depolarization. Next, one or more predetermined cell-containing compartments are aligned with a predetermined position (e.g., aligned with a fluid outlet of an automatic pipette) and an aliquot of a solution containing a compound or mixture of compounds being tested for its ability to modulate NR3B-mediated calcium influx and/or depolarization is delivered to the predetermined compartment(s) with an automatic pipette. Finally, fluorescence emitted by the ion-sensitive indicator in response to an excitation wavelength is measured for a predetermined amount of time, preferably by aligning said cell-containing compartment with a fluorescence detector. Preferably, fluorescence also measured prior to adding NMDA receptor agonist to the cells and/or prior to adding the compound to the wells, to establish e.g., background and/or baseline values for fluorescence.

In accordance with the various assays of the present invention, cells are employed which have ion channels and/or receptors, the activation of which by NMDA agonists results in a change in the level of a cation or anion in the cytoplasm. The cytoplasm of the cells employed are loaded with a fluorescent indicator which is sufficiently sensitive to said ion. By the phrase “sufficiently sensitive fluorescent indicator” is meant a fluorescent compound which, in the presence of, and over a range of physiological concentrations of, a particular ion, is capable of producing distinguishable levels of fluorescence intensity. Preferably, a fluorescent indicator should be able to produce detectably different intensities of fluorescence in response to relatively small changes in ion concentration. The relative intensities of fluorescence when the receptors or ion channels have not been activated, as compared to when the receptors or ion channels have been activated, preferably differ by at least about 50% or more, more preferably by at least about 100-200%.

Any cell which is capable, upon exposure to NMDA agonist, of directly increasing the intracellular concentration of calcium, such as by permitting calcium influx through the NMDA receptor channels, or by causing release of calcium from intracellular stores, may be used in the assay. Preferably neuronal cell lines or cultured neurons are used.

Activation of cellular receptors and/or ion channels (e.g., NMDA-type channels) by incubation with NMDA agonists, may result in a transient increase in the level of intracellular calcium (and/or other ions). The initial increase in calcium may be detected as a rapid increase in fluorescence (e.g., within one to two seconds) after the addition of the NMDA agonist. As shown herein, calcium influx is generally short-lived, but depolarization is longer lasting. Fluorescence levels in the cytoplasm resulting from calcium influx typically increase to a peak value and then typically decline as excess calcium ions are removed by normal cellular mechanisms. The speed at which the fluorescence can be analyzed is important for analysis of the kinetics of the reaction, if it is desired to measure kinetics.

The cells used in the assays of the invention are loaded with a fluorescent indicator which is sufficiently sensitive so as to produce detectable changes in fluorescence intensity in response to changes in the concentration of the ions in the cytoplasm. It is particularly preferred to use a fluorescent indicator which has such sensitivity in the presence of calcium ions. Among the fluorescent indicators which may be employed are the following compounds commercially available from, e.g., Molecular Probes, Inc., Eugene Oreg.: DiBAC₄(3) (B-438), Quin-2 (AM Q-1288), Fura-2 (AM F-1225), Indo-1 (AM I-1226), Fura-3 (AM F-1228), Fluo-3 (AM F-1241), Rhod-2, (AM R-1244), BAPTA (AM B-1205), 5,5′-dimethyl BAPTA (AM D-1207), 4,4′-difluoro BAPTA (AM D-1216), 5,5′-difluoro BAPTA (AM D-1209), 5,5′-dibromo BAPTA (AM D-1213), Calcium Green (C-3011), Calcium Orange (C-3014), Calcium Crimson (C-3017), Fura-5 (F-3023), Fura-Red (F-3020), SBFI (S-1262), PBFI (P-1265), Mag-Fura-2 (AM M-1291), Mag-Indo-1 (AM M-1294), Mag-Quin-2 (AM M-1299), Mag-Quin-1 (AM M-1297), SPQ (M-440), and SPA (S-460).

It is contemplated that each of the individual wells contain the same cell type so that multiple compounds (obtained from different reagent sources in the apparatus or contained within different wells) can be screened and compared for modulating activity with respect to NMDA agonist induction of calcium influx and/or depolarization.

In another of its aspects the invention entails automated antagonist assays. Antagonist assays, including drug screening assays, may be carried out by incubating the cells (e.g., neurons) with NMDA agonists to induce calcium influx and/or depolarization, in the presence and absence of one or more compounds added to the solution bathing the cells in the respective wells of the microtiter plate for an amount of time sufficient for the compound(s) to modulate calcium influx and/or depolarization, and measuring the level of fluorescence in the cells as compared to the level of fluorescence in either the same cell, or substantially identical cell, in the absence of the NMDA agonist. Such assays can be used to identify compounds/chemicals that increase or decrease the function of NR3B subunits in the NMDA receptors. As described above, for some disorders, it may be desirable to identify compounds to increase the activity of the NR3B subunit and in other disorders it may be desirable identify compounds to decrease the activity of the NR3B subunit.

As will be understood by the person of ordinary skill in the art, compounds exhibiting agonist or antagonist activity in an assay of calcium influx or depolarization will either increase or decrease intracellular ion levels (agonist) or inhibit (antagonist) an increase or decrease in the intracellular concentration of ions after incubation of cells with NMDA agonist. It is desirable to measure the amount of agonist or antagonist activity in a linear range of the assay system, such that small but significant increases or decreases in fluorescence relative to control well (e.g., devoid of the test compound) may be observed. It is well within the skill of the art to determine a volume and concentration of a reagent solution which causes a suitable activation response in cells so that modulation of the calcium influx and/or depolarization may be reliably detected.

At a suitable time after addition of the NMDA agonist to initiate calcium influx and/or depolarization, the plate is moved, if necessary, so that the cell-containing assay well is positioned for measurement of fluorescence emission. Because a change in the fluorescence signal may begin within the first few seconds after addition of test compounds, it is desirable to align the assay well with the fluorescence reading device as quickly as possible, with times of about two seconds or less being desirable. In preferred embodiments of the invention, where the apparatus is configured for detection through the bottom of the well(s) and compounds are added from above the well(s), fluorescence readings may be taken substantially continuously, since the plate does not need to be moved for addition of reagent. The well and fluorescence-reading device should remain aligned for a predetermined period of time suitable to measure and record the change in intracellular ion, e.g., calcium, concentration. In preferred embodiments of the invention the fluorescence after activation is read and recorded until the fluorescence change is maximal and then begins to reduce. An empirically determined time period may be chosen which covers the transient rise and fall (or fall and rise) of intracellular ion levels in response to addition of the compound. When the apparatus is configured to detect fluorescence from above the plate, it is preferred that the bottom of the wells are colored black to reduce the background fluorescence and thereby decreases the noise level in the fluorescence reader.

After finishing reading and recording the fluorescence in one well, the just described apparatus steps are repeated with the next well(s) in the series so as to measure pre-reagent fluorescence, add reagent and measure and record the transient change, if any, in fluorescence. The apparatus of the present invention is programmable to begin the steps of an assay sequence in a predetermined first well (or row or column of wells) and proceed sequentially down the columns and across the rows of the plate in a predetermined route through well number n.

In assays of cells treated with NMDA agonist to cause an increase in intracellular calcium ion concentration and/or depolarization, it is preferred that the fluorescence data from replicate wells of cells treated with the same compound are collected and recorded (e.g., stored in the memory of a computer) for calculation of fluorescence and/or intracellular calcium ion concentration.

In assays of compounds that inhibit calcium influx and/or depolarization, the results can be expressed as a percentage of the maximal response caused by NMDA agonist. The maximal fluorescence increase caused by NMDA agonist is defined as being 100% response. For compounds effective for reducing calcium influx and/or depolarization induced by NMDA agonist, the maximal fluorescence recorded after addition of a compound to wells containing NMDA agonist is detectably lower than the fluorescence recorded in the presence of only NMDA agonist.

The fluorescence indicator-based assays of the present invention are thus useful for rapidly screening compounds to identify those that modulate calcium influx and/or depolarization that ultimately results in an altered concentration of ions in the cytoplasm of a cell. For example, the assays can be used to determine functional activity of NR3B subunits upon application of NMDA agonist and can be used to compare activity of NMDA receptors with normal NR3B subunit and NMDA receptors with variant (mutant) NR3B subunits. The assays can preferably be used to determine the effect on functional activity of NR3B subunit containing NMDA receptors, for both normal and variant (mutant) NR3B subunits.

Automation of the fluorescent dye-based assays of the invention can be performed as described in U.S. Pat. No. 6, 057,114. Automation can provide increased efficiency in conducting the assays and increased reliability of the results by permitting multiple measurements over time, thus also facilitating determination of the kinetics of the calcium influx or depolarization effects.

For example, to accomplish rapid compound addition and rapid reading of the fluorescence response, the fluorometer can be modified by fitting an automatic pipetter and developing a software program to accomplish precise computer control over both the fluorometer and the automatic pipetter. By integrating the combination of the fluorometer and the automatic pipetter and using a microcomputer to control the commands to the fluorometer and automatic pipetter, the delay time between reagent addition and fluorescence reading can be significantly reduced. Moreover, both greater reproducibility and higher signal-to-noise ratios can be achieved as compared to manual addition of reagent because the computer repeats the process precisely time after time. Moreover, this arrangement permits a plurality of assays to be conducted concurrently without operator intervention. Thus, with automatic delivery of reagent followed by multiple fluorescence measurements, reliability of the fluorescent dye-based assays as well as the number of assays that can be performed per day are advantageously increased.

The invention further includes nucleic acid or protein microarrays with NR3B polypeptides or nucleic acids encoding such polypeptides. In this aspect of the invention, standard techniques of microarray technology are utilized to assess expression of the NR3B polypeptides and/or identify biological constituents that bind such polypeptides. Protein microarray technology, which is also known by other names including: protein chip technology and solid-phase protein array technology, is well known to those of ordinary skill in the art and is based on, but not limited to, obtaining an array of identified polypeptides or proteins on a fixed substrate, binding target molecules or biological constituents to the polypeptides, and evaluating such binding. See, e.g., G. MacBeath and S. L. Schreiber, Printing Proteins as Microarrays for High-Throughput Function Determination,” Science 289(5485):1760-1763, 2000. Protein arrays, particularly arrays that bind NR3B polypeptides, also can be used for diagnostic applications, such as for identifying subjects that have a condition characterized by NR3B polypeptide expression.

Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (peptide or nucleic acid) on the substrate. Coupling agents or groups on the substrate can be used to covalently link the first nucleotide or amino acid to the substrate. A variety of coupling agents or groups are known to those of skill in the art. Peptide or nucleic acid probes thus can be synthesized directly on the substrate in a predetermined grid. Alternatively, peptide or nucleic acid probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as inkjet or piezo-electric delivery. Probes may be covalently linked to the substrate.

Targets are polypeptides or proteins and may be natural or synthetic. The tissue may be obtained from a subject or may be grown in culture (e.g. from a cell line).

In some embodiments of the invention, one or more control polypeptide or protein molecules are attached to the substrate. Preferably, control polypeptide or protein molecules allow determination of factors such as polypeptide or protein quality and binding characteristics, reagent quality and effectiveness, and analysis thresholds and success.

Nucleic acid microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or CyeS-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol.21, January 1999, the entire contents of which is incorporated by reference herein.

According to the present invention, nucleic acid microarray substrates may include but are not limited to glass, silica, aluminosilicates, borosilicates, metal oxides such as alumina and nickel oxide, various clays, nitrocellulose, or nylon. In all embodiments, a glass substrate is preferred. According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. In one embodiment, preferred probes are sets of one or more of the NR3B nucleic acid molecules set forth herein. Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or olignucleotide to the substrate. These agents or groups may include, for example, amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl-groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, probes are synthesized directly on the substrate in a predetermined grid pattern using methods such as light-directed chemical synthesis, photochemical deprotection, or delivery of nucleotide precursors to the substrate and subsequent probe production.

In another embodiment, the substrate may be coated with a compound to enhance binding of the probe to the substrate. Such compounds include, but are not limited to: polylysine, amino silanes, amino-reactive silanes (Chipping Forecast, 1999) or chromium. In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation. In another embodiment probes are linked to the substrate with heat.

Targets for microarrays are nucleic acids selected from the group, including but not limited to: DNA, genomic DNA, cDNA, RNA, mRNA and may be natural or synthetic. In all embodiments, nucleic acid target molecules from human tissue are preferred. The tissue may be obtained from a subject or may be grown in culture (e.g. from a cell line). In some embodiments, targets for microarrays are proteins/peptides.

In embodiments of the invention one or more control nucleic acid molecules are attached to the substrate. Preferably, control nucleic acid molecules allow determination of factors such as nucleic acid quality and binding characteristics, reagent quality and effectiveness, hybridization success, and analysis thresholds and success. Control nucleic acids may include but are not limited to expression products of genes such as housekeeping genes or fragments thereof.

The invention will be more fully understood by reference to the following examples. These examples, however, are merely intended to illustrate the embodiments of the invention and are not to be construed to limit the scope of the invention.

### EXAMPLES

### Example 1

### Identification of a Novel Glutamate Receptor

Introduction

A TBLASTN search was performed on the published human genomic sequence using the amino acid sequence of the mouse δ2 subunit as a query sequence. This search detected a stretch of genomic sequence with significant homology to members of the glutamate receptor family on a contig from human chromosome 19p13.3 (accession AC004528). This sequence was then used to search the htgs database (unnished high throughput genomic sequences including phases 0, 1 and 2) and the search identified the mouse homologue (AC087114 and AC073805) on chromosome 10. A search of the expressed sequence tag (ESI) database with these sequences identified the following ESTs: accession AL040053 human); AW045848, BE864387, AW048083, BE955769 (mouse); AW525909, BE108608, BE112464 (rat). AW045848 and BE864387 are the same clone sequenced from opposite ends. This clone, which corresponds to the extracellular domain of the putative receptor, was used to screen a mouse spinal cord cDNA library and for in situ hybridization (Clontech, Palo Alto, Calif.).

RT-PCR and in situ Hybridization

RT-PCR was performed with primers spanning predicted exons 1 (CCTCTATAACCTTTCCCGAGG) (SEQ ID NO: 11) and 2 (CTAGAGCAATGTCCTCCCAGG) (SEQ ID NO: 12) of mouse NR3B. The primer set NR1 (5′ primer: GATCCTCGAGCCATGGAGATCGCCTACAAGCGACAC (SEQ ID NO:13), 3′ primer: GATCGGATCCGCATGCTCAGCTCTCCCTATGACGGG (SEQ ID NO: 14) was used as a positive control for the RT-PCR. For in situ hybridizations on brain and spinal cord sections from male mice (C57/BL6, 6 weeks), either ³³P or digoxigenin-labeled RNA probes were used (Simmons et al., 1989; Lu et al., in press). Serotonin was immunohistochemically detected with anti-serotonin antibody (Incstar, Stillwater, Minn.).

Electrophysiology in HEK293

To express the receptors in HEK293 cells, the cells were transfected with 1 μg of each plasmid unless otherwise stated (Shi et al., 1999). The NR1 and NR2A were tagged with green fluorescent protein (GFP) on the extracellular domain to facilitate the identification of transfected cells. Such constructs were shown to preserve the properties of the glutamate receptor (Shi et al., 1999). NMDA receptor mediated current was recorded in the presence of 10 μM glycine. AMPA receptor current was recorded as described (Shi et al., 1999). Pipette solutions contained (in mM) Cs-methanesulfonate 110; CsCl 30; NaCl 4; HEPES 10; EGTA 5; CaCl₂ 0.5 adjusted to pH 7.3 with CsOH. 1 mM glutamate (for NMDA receptor) or kainate (for AMPA receptor) was pressure (1.0 PSI) applied through a puffer pipette positioned ˜10 μm to the cells. A program based on Igor (WaveMetrics, Inc., Lake Oswego, Oreg.) was used to acquire data on a Macintosh computer.

Rat Nucleic Acid and Polypeptide Sequences.

The following NR3B nucleic acid and amino acid sequences have been determined in rat: SEQ ID NOs: 5-11. SEQ ID NOs: 5 and 8 correspond to EST clone UI-R-B01-aiw-b-09-o-uI (Accession No: AW525909), SEQ ID NOs: 6 and 9 correspond to EST clone UI-R-CAO-axg-c-09-0-UI (Accession No.: BE108608) and SEQ ID NOS: 7 and 10 correspond to UI-R-BJ2-avi-a-03-0-UI (Accession No: BE112464).

Results

Identification and Cloning of a New Mouse Glutamate Receptor

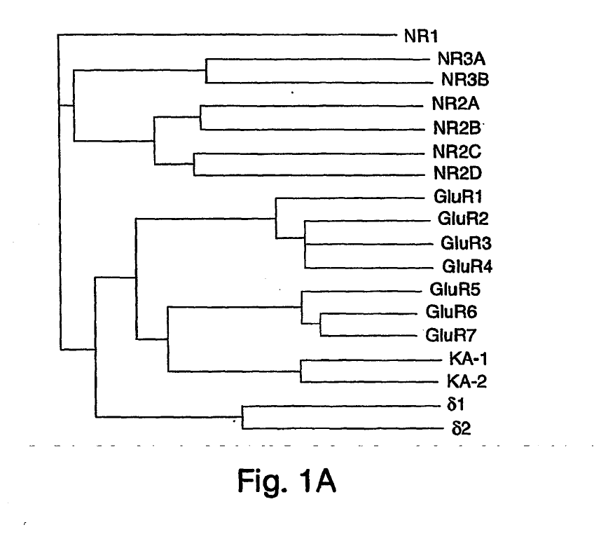

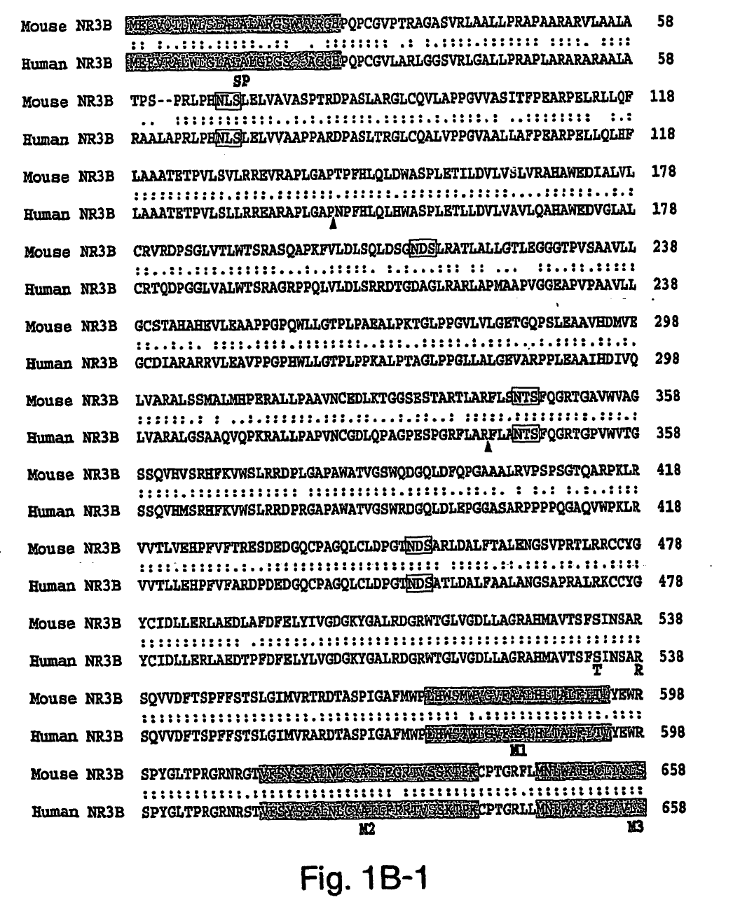

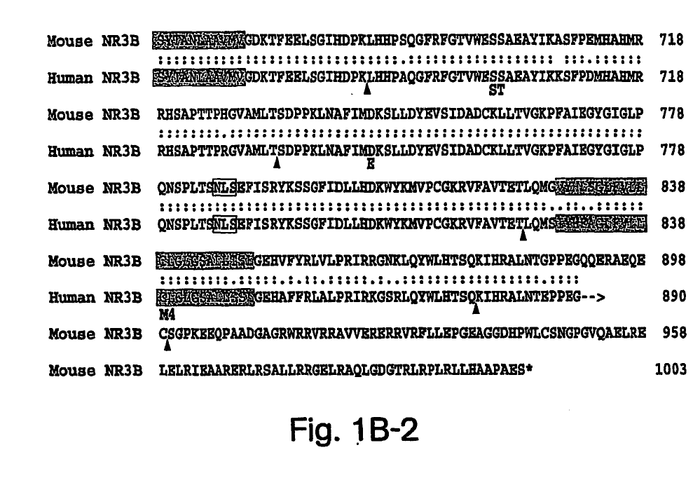

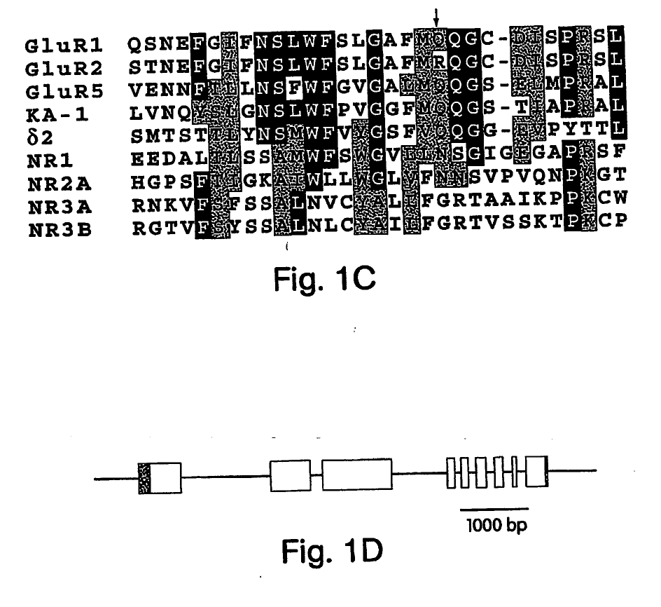

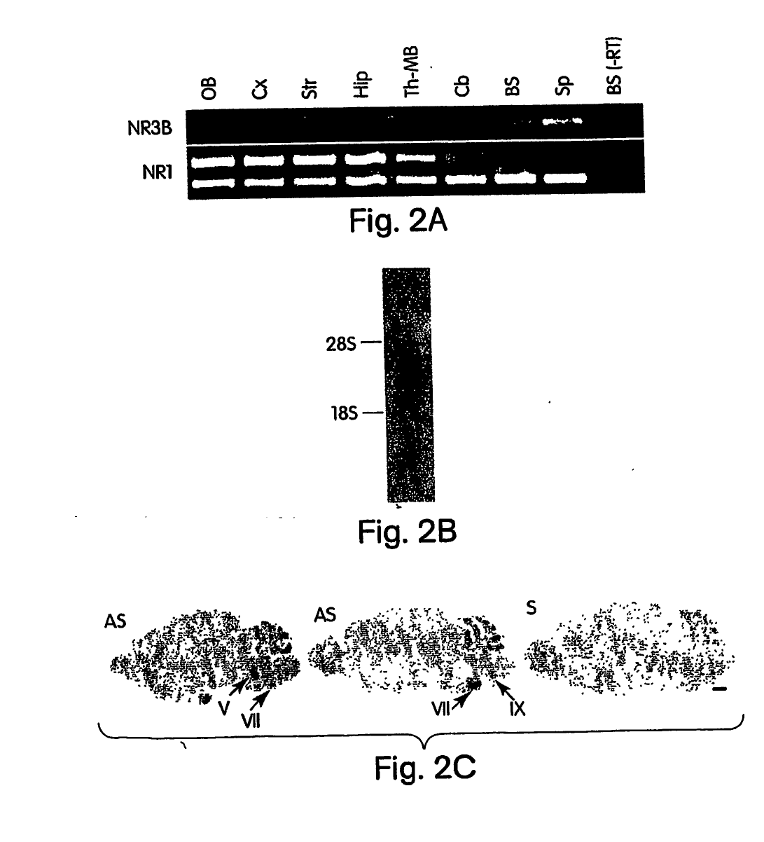

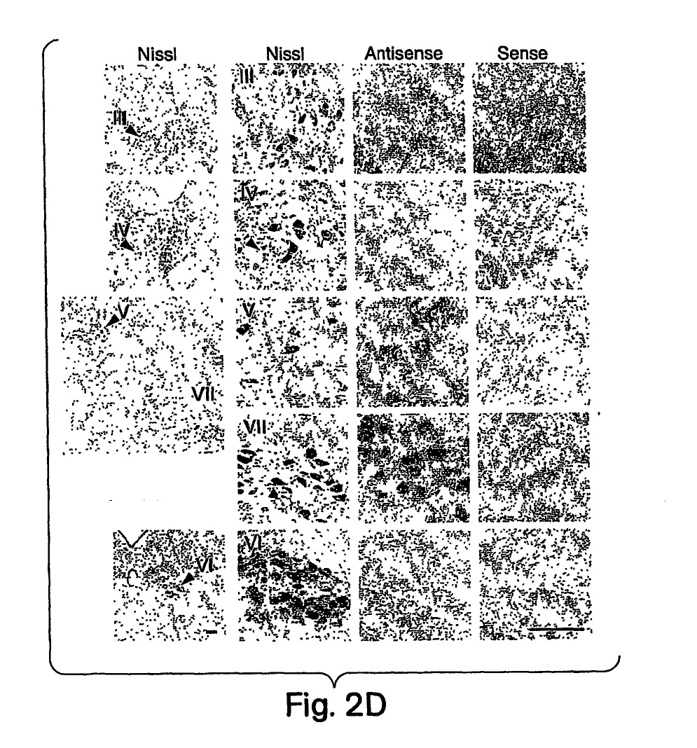

A unique sequence with significant homology to the glutamate receptor family (FIG. 1A) was identified by BLAST search of the published human and mouse genomic sequences. Transcript levels and the tissue distribution of mRNA were examined by RT-PCR of mouse RNA using a primer set spanning the predicted exons 1 and 2 (FIG. 2A). This revealed a band of the expected size for spliced product in brainstem and spinal cord. A fainter band was also detected in the cerebellum. By use of other primer sets covering exons 2 and 3, a similar pattern of expression was detected. More rostral structures did not show any significant expression of this transcript, whereas RT-PCR for the NR1 subunit carried out in parallel showed positive bands in all brain regions. When reverse transcriptase was omitted from the reaction (-RT), the band was not detected, ruling out amplification from contaminating genomic DNA. A further BLAST search identified EST clones from human, mouse, and rat. When the mouse EST clone AW045848 was used to probe polyA+RNA from mouse spinal cord, it hybridized to a band at ˜3.5 kb (FIG. 2B). This clone was used to screen a mouse spinal cord cDNA library. The screening of approximately 10⁶ clones yielded three positive clones, one of which contained full-length cDNA (accession AF396649).

Structure of-the NR3B gene, cDNA, and Protein Product

Sequence analysis of the full-length mouse clone revealed a cDNA of 3283 bp encoding a polypeptide of 1003 amino acids (FIG. 1B). The cDNA was found to be encoded by at least nine exons in the mouse genome spanning approximately 6.5 kb (FIG. 1D). A TATA box was found in the genomic sequence at −339 from the start codon ATG whereas the cDNA starts at −185, suggesting that there exists 5′-untranslated sequence beyond the CDNA. All nucleotides in the genomic sequence and the cDNA were found to be identical, indicating that RNA editing does not occur in this gene. The predicted polypeptide has a significant similarity to NR3A (51% identity), and therefore it was named NR3B (FIG. 1A). A glutamate receptor member named δ-2 has been reported in abstract form but due to a lack of detailed information, the identity of NR3B with δ-2 is unknown (Sevarino et al., 1996). The human sequence was assembled from the genomic sequence deposited in GenBank (FIG. 1B). The C-terminus of the human clone loses homology after Gly890, which is followed by a possible splice donor site (GGGT), indicating there may be one or more additional exon(s) in the human sequence. All the other exon-intron boundaries are preserved between the two species and conform to a consensus splice donor-acceptor site (GT-AG). The homology between the human and mouse is 81.3%.

A full-length human NR3B CDNA sequence (SEQ ID NO: 3) that corresponds to the human NR3B sequence identified through homology to the mouse NR3B sequence, is disclosed in PCT Application WO 01/44473. SEQ ID NO: 4 is the polypeptide encoded by the human NR3B nucleic acid (SEQ ID NO: 3) and the polypeptide sequence (Accession no: AAC12680) corresponds to the human NR3B polypeptide identified through homology to the mouse NR3B polypeptide.

NR3B has a transmembrane topology typical of glutamate receptor with an N-terminal signal peptide and four membrane-associated regions. Five (in mouse) or four (in human) consensus sequences for N-glycosylation sites are found on the N-terminal domain and one (for both species) at the loop between the third and fourth membrane-associated regions. The intracellular C-terminus of mouse NR3B has three threonines and five serines, which may serve as regulatory phosphorylation sites. The C-terminus ends with Ala-Glu-Ser, which does not conform to the consensus PDZ domain protein-binding site sequence typical of other glutamate receptor family members (Songyang et al., 1997; Sheng and Sala, 2001).

The amino acid residues forming the glutamate-binding pocket have been elucidated by structural studies of the crystallized ligand-binding domain of GluR2 (Armstrong and Gouaux, 2000). Importantly, the 2-carboxyl group of glutamate binds to the guanidium group of Arg485 and the NH group of Thr480 and the 2-amino group interacts with the carboxyl group of Glu705 in GluR2. These residues can be mapped precisely on the sequence of NR3B with Thr480 of GluR2 corresponding to Ser533 of NR3B, Arg485 to Arg538, and Glu705 to Asp745. Other amino acids forming the putative ligand-binding pocket are also well conserved (FIG. 1B), indicating that NR3B itself is likely to bind to glutamate.

Expression of NR3B is Limited to Motoneurons

The RT-PCR revealed that NR3B is expressed selectively in a limited number of brain regions. Consistently, in situ hybridization of a sagittal section of a mouse brain detected signal in limited regions in the brainstem (FIG. 2C). A counterstaining of the sections showed that these are the trigeminal motor (V), facial (VII), and ambiguus nuclei (IX). A higher magnification of these structures revealed hybridization signals in motoneurons with large cell bodies (FIG. 2D). In contrast to this, the signal was significantly weaker in motoneurons in the nuclei controlling the external ocular muscles (oculomotor, III; trochlear, IV, abducens, VI, FIG. 2D).

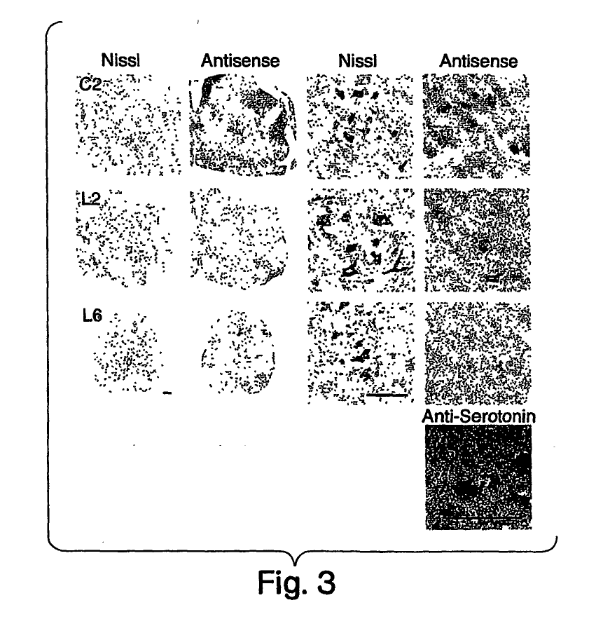

In the spinal cord, the signal was also detected in the motoneurons in the anterior horn (FIG. 3). However, in the L6 level, the signal intensity of NR3B in motoneurons was significantly weaker than higher levels though the presence of motoneurons at this level was confirmed by Nissl staining. These motoneurons were classified as those in Onuf's nucleus that controls external anal and urethral sphincters, based on plexus formation of serotonin immunoreactive fibers on cell body (FIG. 3 bottom) (Micevych et al., 1986) and a comparison with the cytoarchitecture of the mouse spinal cord (Sidman et al., 1971) as well as with the previous studies on rat counterpart.

NR3B acts as a Dominant-Negative Subunit

The electrophysiological properties of NR3B were tested by expressing NR3B in HEK293 cells. A conventional NMDA receptor channel is formed by a heteromeric combination of NR1, which is a key subunit for functionality, and at least one NR2 subunit, which is modulatory (Dingledine et al., 1999). NR3B cannot substitute for either of these subunits as expression of NR3B alone (n=6) or coexpression with NR1 (n=6) or NR2A (n=5) did not result in electrophysiologically functional channels whereas coexpression of NR1 and NR2A gave a robust current (FIG. 4). However, coexpression of all three subunits markedly depressed the whole-cell current compared with the combination of NR1 and NR2A (FIG. 4A, C). Increasing the amount of NR3B versus the other subunits had a stronger suppressive effect (FIG. 4C). NR3B act as a dominant-negative subunit and suppresses glutamate-induced whole-cell current in cells coexpressing NR1 and NR2A. The distribution of averaged responses obtained at +60 mV in Mg²⁺-free solution is shown in a cumulative plot. (FIG. 4C). Increasing the amount of NR3B plasmid (0, 1, and 3 μg) versus other subunits (each 1 μg) caused a concomitant decrease in current amplitude. Statistical significances were: 1:1:0 vs. 1:1:1, p<0.05; 1:1:0 vs. 1:1:3, p<0.01; 1:1:1 vs. 1:1:3, p<0.01. AMPA receptor mediated current was unchanged by the coexpression with NR3B. GluR2-(R586Q), a point mutation of the GluR2 subunit of AMPA receptors that gives a large current, was used because it was empirically known that this construct gives reliable response. This is not due to a non-specific reduction of the polypeptide expression level as a direct quantification of expression levels of NR1 and NR2A tagged with GFP using fluorescence as a measure did not show any significant difference in the presence or absence of NR3B. NR1 and NR2A tagged with GFP were individually expressed with NR3B and their expression levels were measured by the fluorescence intensity. The statistical significance in this figure was assessed by Kolmogorov-Smirnov test. (FIG. 4D). The effect of NR3B is specific to NMDA receptor as the AMPA receptor mediated current was not changed by the presence of NR3B (FIG. 4C). The sensitivity of the remaining current to Mg²⁺ block was not affected by the presence of NR3B subunit (FIG. 4B). The remaining current in NR3B-expressing cells exhibits a Mg²⁺ block indistinguishable from cells not expressing NR3B. The slight block at −60 mV and −80 mV may be due to residual Mg²⁺ (FIG. 4B).

### References

Armstrong N, Gouaux E (2000) Mechanisms for activation and antagonism of an AMPA-sensitive glutamate receptor: crystal structures of the GluR2 ligand binding core. Neuron 28:165-181.
Carmignoto G, Vicini S (1992) Activity-dependent decrease in NMDA receptor responses during development of the visual cortex. Science 258:1007-1011.
Ciabarra A M, Sullivan J M, Gahn L G, Pecht G, Heinemann S, Sevarino K A (1995) Cloning and characterization of c-1: a developmentally regulated member of a novel class of the ionotropic glutamate receptor family. J Neurosci 15:6498-6508.
Das S, Sasaki Y F, Rothe T, Premkumar L S, Takasu M, Crandall J E, Dikkes P, Conner D A, Rayudu P V, Cheung W, Chen H S, Lipton S A, Nakanishi N (1998) Increased NMDA current and spine density in mice lacking the NMDA receptor subunit NR3A Nature 393:377-381.
Dingledine R, Borges K, Bowie D, Traynelis S F (1999) The glutamate receptor ion channels. Pharmacol Rev 51:7-61.
Hollmann M, Heinemann S (1994) Cloned glutamate receptors. Annu Rev Neurosci 17:31-108.
Lu H, Ozawa H, Nishi M, Ito T, Kawata M (in press) Serotonergic neurones in the dorsal raphe nucleus that project into the medial preoptic area contain oestrogen receptor b. J Neuroendocrinol.
Micevych P E, Coquelin A, Arnold A P (1986) Immunohistochemical distribution of substance P, serotonin, and methionine enkephalin in sexually dimorphic nuclei of the rat lumbar spinal cord. J Comp Neurol 248:235-244.
Nakanishi S, Nakajima Y, Nomura A, Masu M, Iwakabe H, Hayashi Y, Yokoi M (1996) Functions and roles of glutamate receptors in synaptic transmission and plasticity. Cold Spring Harb Symp Quant Biol 61:67-75.
Perez-Otano I, Schulteis C T, Contractor A, Lipton S A, Trimmer J S, Sucher N J, Heinemann S F (2001) Assembly with the NR1 subunit is required for surface expression of NR3A-containing NMDA receptors. J Neurosci 21:1228-1237.
Quinlan E M, Philpot B D, Huganir R L, Bear M F (1999) Rapid, experience-dependent expression of synaptic NMDA receptors in visual cortex in vivo. Nat Neurosci 2:352-357.
Seeburg P H (1993) The TiPS/TINS lecture: the molecular biology of mammalian glutamate receptor channels. Trends Pharmacol Sci 14:297-303.
Sevarino K A, Ciabarra A M, Forcina M S (1996) c-1 and c-2, members of a novel class of the glutamate receptor superfamily. Soc Neurosci Abstr 22:592.
Sheng M, Sala C (2001) PDZ domains and the organization of supramolecular complexes. Annu Rev Neurosci 24:1-29.
Shi S H, Hayashi Y, Petralia R, Zaman S, Wenthold R, Svoboda K, Malinow R (1999) Rapid Spine Delivery and Redistribution of AMPA Receptors after Synaptic NMDA Receptor Activation. Science 284:1811-1816.
Sidman R L, Angevine Jr J B, Pierce E T (1971) Atlas of the mouse brain and spinal cord.Cambridge, Mass.:A Commonwealth Fund Book, Harvard University Press.
Simmons D M, Arriza J L, Swanson L W (1989) A complete protocol for in situ hybridization of messenger RNAs in brain and other tissues with radiolabeled single-stranded RNA probes. J Histotech 12:169-181.
Songyang Z, Fanning A S, Fu C, Xu J, Marfatia S M, Chishti A H, Crompton A, Chan A C, Anderson J M, Cantley L C (1997) Recognition of unique carboxyl-terminal motifs by distinct PDZ domains. Science 275:73-77.
Sucher N J, Akbarian S, Chi C L, Leclerc C L, Awobuluyi M, Deitcher D L, Wu M K, Yuan J P, Jones E G, Lipton S A (1995) Developmental and regional expression pattern of a novel NMDA receptor-like subunit (NMDAR-L) in the rodent brain. J Neurosci 15:6509-6520.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All references, publications and patents disclosed herein are incorporated by reference in their entirety.

1. An isolated nucleic acid molecule selected from the group consisting of:
(a) nucleic acid molecules which hybridize under high stringency conditions to a nucleic acid molecule having a nucleotide sequence set forth as SEQ ID NO: 1, providing that no more than about 18% of the nucleotides are changed from SEQ ID NO: 1,
(b) nucleic acid molecules that differ from the nucleic acid molecules of (a) in codon sequence due to the degeneracy of the genetic code, and
(c) complements of (a) or (b), wherein the nucleic acid molecules or complements thereof code for a mouse NR3B NMDA receptor subunit.

2. The isolated nucleic acid molecule of claim 1, wherein the isolated nucleic acid molecule comprises a nucleic acid sequence set forth as: SEQ ID NO: 1.

3. An isolated nucleic acid molecule selected from the group consisting of:
(a) fragment of nucleotides 1-3290 of SEQ ID NO: 1 between 24 and 3289 nucleotides in length, providing that no more than about 18% of the nucleotides are changed from SEQ ID NO:1, and
(b) complements of (a).

4. An expression vector comprising the isolated nucleic acid molecule of claim 1 operably linked to a promoter.

5. A host cell transformed or transfected with the expression vector of claim 4.

6. A transgenic non-human animal comprising the expression vector of claim 4.

7. An isolated polypeptide encoded by the isolated nucleic acid molecule of claim 1.

8. The isolated polypeptide of claim 7, wherein the isolated polypeptide comprises an amino acid sequence selected from the group consisting of SEQ ID NO: 2 and a fragment or functional variant of SEQ ID NO:2.

9. A binding polypeptide that selectively binds the isolated polypeptide of claim 7, wherein the binding polypeptide is an antibody or antigen-binding fragment thereof.

10. The binding polypeptide of claim 9, wherein the binding polypeptide selectively binds the polypeptide sequence of SEQ ID NO: 2.

11. A composition comprising a molecule selected from the group consisting of:
(a) the nucleic acid of claim 1,
(b) the polypeptide encoded by the isolated nucleic acid molecule of claim 1, and
(c) the binding polypeptide of claim 9, and
a pharmaceutically acceptable carrier.

12. A method for making a medicament, comprising:
placing in a pharmaceutically acceptable carrier, a molecule selected from the group consisting of: (a) the isolated nucleic acid molecules of claim 1, (b) the isolated polypeptide of claim 7, and (c) the binding polypeptides of claim 9.

13. The method of claim 12, wherein the step of placing comprises placing a therapeutically effective amount of the molecule selected from the group in the pharmaceutically acceptable carrier to form one or more doses.

14. A method of making a glutamate receptor in vitro, comprising introducing glutamate receptor nucleic acids into a cell, wherein the glutamate receptor nucleic acids encode a NR3B polypeptide and wherein the nucleic acids are as claimed in claim 1.

15. The method of claim 14, wherein the NR3B subunit is encoded by the nucleotide sequence set forth as SEQ ID NO: 1.

16. A method for diagnosing a motor neuron disorder characterized by aberrant expression of a NR3B molecule, comprising:
detecting expression of a NR3B molecule in a first biological sample obtained from a subject,
wherein a difference in expression level of the NR3B molecule compared to expression level a NR3B molecule in a control sample indicates that the subject has a motor neuron disorder characterized by aberrant expression of a NR3B molecule, and wherein the NR3B molecule is a nucleic acid molecule as claimed in claim 1 or is a polypeptide encoded by a nucleic acid molecule as claimed in claim 1.

17. The method of claim 16, further comprising the steps of:
detecting expression of a NR3B molecule in a second biological sample obtained from the subject at a time subsequent to the first biological sample, and
comparing the expression of the NR3B molecule in the first biological sample and the second biological sample as an indication of the onset, progression, or regression of the motor neuron disorder.

18. The method of claim 17, wherein a decrease in expression level of the NR3B in the second biological sample compared to the expression level in the NR3B in the first biological sample indicates progression of the motor neuron disorder characterized by aberrant expression of NR3B.

19. (canceled)

20. The method of claim 17, wherein an increase in expression level of the NR3B in the second biological sample compared to the expression level in the NR3B in the first biological sample indicates regression of the motor neuron disorder characterized by aberrant expression of NR3B.

21-22. (canceled)

23. The method of claim 16, comprising detecting expression of a NR3B nucleic acid molecule.

24. The method of claim 23, wherein the NR3B nucleic acid molecule comprises the nucleic acid sequence set forth as SEQ ID NO: 1 or fragment thereof.

25. The method of claim 16, comprising detecting expression of a NR3B polypeptide.

26. The method of claim 25, wherein the NR3B polypeptide is set forth as SEQ ID NO: 2 or fragment thereof.

27. The method of claim 16, wherein detecting comprises contacting the biological sample with an agent that selectively binds the NR3B molecule.

28. The method of claim 27, wherein the NR3B molecule is a nucleic acid and wherein the agent that selectively binds the NR3B molecule is a nucleic acid selected from the group of nucleic acid molecules comprising the nucleotide sequences that hybridize to SEQ ID NO: 1 under high stringency conditions.

29. The method of claim 27, wherein the NR3B molecule is a polypeptide and wherein the agent that selectively binds the NR3B molecule is a binding polypeptide selected from the group of binding polypeptides that selectively bind to SEQ ID NO: 2.

30. (canceled)

31. A method for evaluating the effect of candidate pharmacological compounds on expression of an NR3B subunit of a glutamate receptor, comprising:
administering a candidate pharmaceutical agent to a subject;
determining the effect of the candidate pharmaceutical agent on the expression level of NR3B relative to the expression level of NR3B in a subject to which no candidate pharmaceutical agent is administered, wherein a relative increase or relative decrease in the expression level of NR3B indicates the effect of the candidate pharmaceutical compound on the expression of the NR3B subunit of the glutamate receptor, and wherein NR3B is a nucleic acid molecule as claimed in claim 1 or is a polypeptide encoded by a nucleic acid molecule as claimed in claim 1.

32-33. (canceled)

34. A method for evaluating the effect of candidate pharmacological compounds on the expression of a NR3B subunit of a glutamate receptor, comprising:
contacting a candidate pharmaceutical agent with a NR3B subunit expressing cell or tissue sample;
determining the effect of the candidate pharmaceutical agent on the expression level of NR3B relative to the expression level of NR3B in a NR3B subunit expressing cell or tissue sample not contacted with the candidate pharmaceutical agent, wherein a relative increase or relative decrease in the expression level of NR3B indicates the effect of the candidate pharmaceutical compound on the expression of NR3B subunit of a glutamate receptor and wherein NR3B is a nucleic acid molecule as claimed in claim 1 or is a polypeptide encoded by a nucleic acid molecule as claimed in claim 1.

35-36. (canceled)

37. A method for diagnosing a motor neuron disorder characterized by aberrant function of a NR3B molecule, comprising:
detecting function of a NR3B molecule in a first biological sample obtained from a subject,
wherein a difference in function of the NR3B molecule compared to a NR3B molecule in a control sample indicates that the subject has a motor neuron disorder characterized by aberrant function of a NR3B molecule, and wherein the NR3B molecule is a nucleic acid molecule as claimed in claim 1 or is a polypeptide encoded by a nucleic acid molecule as claimed in claim 1.

38. The method of claim 37, further comprising the steps of:
detecting function of a NR3B molecule in a second biological sample obtained from the subject at a time subsequent to the first biological sample, and
comparing the function of the NR3B molecule in the first biological sample and the second biological sample as an indication of the onset, progression, or regression of the motor neuron disorder.

39. The method of claim 38, wherein a decrease in function level of the NR3B in the second biological sample compared to the function level in the NR3B in the first biological sample indicates progression of the motor neuron disorder characterized by aberrant function of NR3B.

40. (canceled)

41. The method of claim 38, wherein an increase in function level of the NR3B in the second biological sample compared to the function level in the NR3B in the first biological sample indicates regression of the motor neuron disorder characterized by aberrant expression of NR3B.

42-43. (canceled)

44. The method of claim 37, comprising detecting function of a NR3B nucleic acid molecule.

45. The method of claim 44, wherein the NR3B nucleic acid molecule comprises a nucleic acid sequence set forth as SEQ ID NO: 1 or fragment thereof.

46. The method of claim 37, comprising detecting function of a NR3B polypeptide.

47. The method of claim 46, wherein the NR3B polypeptide is set forth as SEQ ID NO: 2 or fragment thereof.

48. The method of claim 37, wherein detecting comprises determining the cation passage through an NMDA receptor channel.

49. The method of claim 48, wherein the cation flux is determined with a method selected from the group consisting of: electrophysiological recording, drug screening assays, and ion-flux measurement.

50. (canceled)

51. A method for evaluating the effect of candidate pharmacological compounds on function of an NR3B subunit of a glutamate receptor, comprising:
administering a candidate pharmaceutical agent to a subject that expresses a glutamate receptor containing a functional NR3B subunit;
detecting the function of the NR3B subunit of the glutamate receptor,
determining the effect of the candidate pharmaceutical agent on the function level of NR3B relative to the function level of NR3B in a subject to which no candidate pharmaceutical agent is administered, wherein a relative increase or relative decrease in the function level of NR3B indicates the effect of the candidate pharmacological compound on the function of the NR3B subunit of the glutamate receptor, and wherein NR3B is a nucleic acid molecule as claimed in claim 1 or is a polypeptide encoded by a nucleic acid molecule as claimed in claim 1.

52-53. (canceled)

54. The method of claim 51, wherein detecting comprises determining the cation passage through an NMDA receptor channel.

55. The method of claim 51, wherein the cation flux is determined with a method selected from the group consisting of: electrophysiological recording, drug screening assays, and ion-flux measurement.

56. A method for evaluating the effect of candidate pharmacological compounds on function of a NR3B subunit of a glutamate receptor, comprising:
contacting a glutamate receptor sample with a candidate pharmaceutical agent;
detecting the function of the NR3B subunit of the glutamate receptor,
determining the effect of the candidate pharmaceutical agent on the function level of NR3B relative to the function level of NR3B in a glutamate receptor sample not contacted with the candidate pharmaceutical agent, wherein a relative increase or relative decrease in the function level of NR3B indicates the effect of the candidate pharmacological agent on the function of NR3B subunit of the glutamate receptors, and wherein NR3B is a nucleic acid molecule as claimed in claim 1 or is a polypeptide encoded by a nucleic acid molecule as claimed in claim 1.

57-58. (canceled)

59. The method of claim 56, wherein detecting comprises determining the cation passage through an NMDA receptor channel.

60. The method of claim 56, wherein the cation flux is determined with a method selected from the group consisting of: electrophysiological recording, drug screening assays, and ion-flux measurement.

61. A kit for diagnosing a motor neuron disorder associated with aberrant expression of a NR3B molecule, comprising:
one or more nucleic acid molecules that hybridize to a NR3B nucleic acid molecule under high stringency conditions and
instructions for the use of the nucleic acid molecules in the diagnosis of a motor neuron disorder associated with aberrant expression of a NR3B molecule, wherein the NR3B nucleic acid molecule is a nucleic acid molecule as claimed in claim 1.

62. The kit of claim 61, wherein the one or more nucleic acid molecules are a first primer and a second primer and, wherein the first primer and the second primer are constructed and arranged to selectively amplify at least a portion of an isolated NR3B nucleic acid molecule comprising SEQ ID NO: 1.

63. A kit for diagnosing a NR3B-associated motor neuron disorder in a subject comprising:
one or more binding polypeptides that selectively bind to a NR3B polypeptide, and
instructions for the use of the binding polypeptides in the diagnosis of a motor neuron disorder associated with aberrant expression of a NR3B molecule, wherein the NR3B polypeptide is encoded by a nucleic acid molecule as claimed in claim 1.

64. (canceled)

65. The kit of claim 63, wherein the NR3B polypeptide is encoded by a nucleic acid comprising a nucleotide sequence set forth as SEQ ID NO: 1.

66. A method for treating a subject with a motor neuron disorder characterized by decreased expression of a NR3B molecule, comprising
administering to the subject an amount of a NR3B nucleic acid molecule effective to increase expression of a NR3B polypeptide and treat the motor neuron disorder, wherein the NR3B nucleic acid molecule is a nucleic acid molecule as claimed in claim 1.

67. A method for treating a subject with a motor neuron disorder characterized by decreased expression of a NR3B polypeptide, comprising
administering to the subject an amount of a NR3B polypeptide effective to treat the motor neuron disorder, wherein the NR3B polypeptide is encoded by a nucleic acid molecule as claimed in claim 1.

68. A method for treating a subject with a motor neuron disorder characterized by increased expression of a NR3B nucleic acid molecule, comprising
administering to the subject an amount of an antisense molecule to a NR3B nucleic acid molecule effective to treat the motor neuron disorder, wherein the NR3B nucleic acid molecule is a nucleic acid molecule as claimed in claim 1.

69. A method for treating a subject with a motor neuron disorder characterized by increased expression of a NR3B polypeptide, comprising:
administering to the subject an amount of a NR3B polypeptide binding polypeptide effective to treat the motor neuron disorder, wherein the NR3B polypeptide is a polypeptide encoded by a nucleic acid molecule as claimed in claim 1.

70. (canceled)

71. A method for treating a subject with a motor neuron disorder characterized by decreased function of a NR3B molecule, comprising
administering to the subject an amount of a NR3B nucleic acid molecule effective to treat the motor neuron disorder, wherein the NR3B nucleic acid molecule is a nucleic acid molecule as claimed in claim 1.

72. A method for treating a subject with a motor neuron disorder characterized by decreased function of a NR3B polypeptide, comprising
administering to the subject an amount of a NR3B polypeptide effective to treat the motor neuron disorder, wherein the NR3B polypeptide is a polypeptide encoded by a nucleic acid molecule as claimed in claim 1.

73. A method for treating a subject with a motor neuron disorder characterized by increased function of a NR3B nucleic acid molecule, comprising
administering to the subject an amount of an antisense molecule to a NR3B nucleic acid molecule effective to treat the motor neuron disorder, wherein the NR3B nucleic acid molecule is a nucleic acid molecule as claimed in claim 1.

74. A method for treating a subject with a motor neuron disorder characterized by increased function of a NR3B polypeptide, comprising:
administering to the subject an amount of a NR3B polypeptide binding polypeptide effective to treat the motor neuron disorder, wherein the NR3B polypeptide is a polypeptide encoded by a nucleic acid molecule as claimed in claim 1.

75. (canceled)

76. A method for producing a NR3B polypeptide or fragment thereof, comprising:
providing an isolated NR3B nucleic acid molecule operably linked to a promoter, wherein the NR3B nucleic acid molecule encodes the NR3B polypeptide or fragment thereof, and
expressing the NR3B nucleic acid molecule in an expression system, wherein the NR3B nucleic acid molecule is a nucleic acid molecule as claimed in claim 1.

77. The method of claim 76, further comprising:
isolating the NR3B polypeptide or a fragment thereof from the expression system.

78. The method of claim 76, wherein the NR3B nucleic acid molecule is set forth as SEQ ID NO: 1.

79. A method for making a NR3B polypeptide comprising:
culturing the host cell of claim 5, and isolating the NR3B polypeptide from the culture.

80. A method for preparing a model of a motor neuron disease characterized by aberrant expression of a NR3B molecule, comprising introducing into a cell, a NR3B molecule wherein the NR3B molecule is a nucleic acid molecule as claimed in claim 1 or is a polypeptide encoded by a nucleic acid molecule as claimed in claim 1.

81. (canceled)

82. The method of claim 80, wherein the NR3B molecule is a NR3B nucleic acid molecule set forth in SEQ ID NO: 1.

83. The method of claim 80, wherein the NR3B molecule is a NR3B polypeptide set forth in SEQ ID NO: 2.

84-85. (canceled)

86. A method for preparing an animal model of a motor neuron disorder characterized by aberrant function of a NR3B molecule, comprising:
introducing into a non-human subject, an aberrant NR3B molecule; and
detecting expression of the aberrant NR3B molecule in a first biological sample obtained from the non-human subject, wherein the NR3B molecule is a nucleic acid molecule as claimed in claim 1 or is a polypeptide encoded by a nucleic acid molecule as claimed in claim 1.

87-92. (canceled)

93. A method for preparing a non-human animal model of a motor neuron disorder characterized by reduced expression of a NR3B molecule, comprising
administering to a non-human subject an effective amount of an anti-sense molecule to a NR3B nucleic acid molecule to reduce expression of the NR3B nucleic molecule in the non-human subject.

94. The method of claim 93, wherein the NR3B molecule is a nucleic acid molecule selected from the group containing SEQ ID NO: 1 and SEQ ID NO:3.

95. A method for preparing a non-human animal model of a motor neuron disorder characterized by reduced expression of a NR3B molecule, comprising
administering to a non-human subject an effective amount of a binding polypeptide to a NR3B polypeptide to reduce expression of the NR3B polypeptide in the non-human subject.

96. (canceled)

97. The method of claim 95, wherein the NR3B molecule a nucleic acid molecule selected from the group containing SEQ ID NO: 1 and SEQ ID NO:3.


SEQUENCE LISTING (23 sequences)

SEQ ID NO: 1 - DNA, 3290 bp, Mus musculus
tgctctgggagcttcagtctgagcagtggaaggcatcacccaactctgccctgcgtggcacagcaaacctaggcagcggcccagctcctcctcctgtggacacccggaccggcaggaggaacgagagcggggctggctgggatgtctccttaccctcgcacagcacagtggtaacttccatcgggatggagtgtgtgcagacgctgtggctcagcctggccctggcgctggcgcgagggtcctgggtggtgcgcggtcaccctcagccctgcggggttcccacgcgcgccggggcctccgtgcgcctggctgcgctcctaccccgggcgcccgccgcccgcgcccgcgtcctagccgccctggccaccccttccccgcggctgccgcacaacctgagtctggagctagtggccgtcgcgtccccaacccgggaccccgcgtcgctggcccgaggtctgtgccaggttctggcaccgcccggcgtggtggcctctataacctttcccgaggcgcggcctgagctacggctattgcagttcctggcagctgccacagagaccccggtgctgagcgtcctacggagggaggtgcgcgcgcccctcggagctccgaccccgttccacctgcagctggactgggctagtcccctggagaccatcctggatgtgctggtgtccctggtacgggcacacgcctgggaggacattgctctagtgctctgtcgtgtccgggaccccagtggcctggtgacactctggaccagccgtgctagccaggctccaaagtttgtgctggacctgagccagttggacagcgggaatgacagccttcgggctacactggccctgctggggacgctggaaggagggggaacccccgtgtctgcagccgtcctcctgggctgcagcactgcccatgcacatgaggtcctagaggcagcaccaccgggtccccagtggctgctgggcacaccactgcccgccgaggcactgcccaaaaccggtctgccccctggggtgttggtgctgggggaaaccgggcagccttccctggaagctgccgtccacgacatggtggagcttgtggctcgggcactcagcagcatggccctcatgcacccagagcgggccctgcttccagcggcagtaaattgtgaggacctgaaaacgggcggctctgagtcaacagcacgcaccttggctaggtttctgagcaacacctcatttcagggccgcacaggggctgtgtgggtggcaggctcctctcaggtgcatgtgtctcggcatttcaaggtatggagcttacgcagggacccgctgggtgccccagcctgggcaacagtaggcagctggcaggatggacagctggacttccagccaggggcggctgctctccgagttccatctccatctggcacccaggcccggccaaagctgcgagtggtaactctggtggaacacccatttgtgttcaccagggaatctgatgaagatgggcagtgcccggctgggcagctgtgtctagacccaggcaccaatgactcggccaggctggatgcgctcttcactgcattggagaatggctccgtgcctcgcaccctgagaagatgctgttatggctactgcattgacctgctggagcggctggccgaggacctggcctttgactttgagctctatattgtgggggatggcaagtacggggccctgcgtgatggacgctggacaggcctggtgggtgacctgctggccggtcgggcacacatggccgtgaccagcttcagtatcaactcggctcgctcccaggtggtggatttcaccagccctttcttctccaccagcctgggcatcatggtgcgcacgcgagatacagcctcacccattggggctttcatgtggcccctgcactggtccatgtgggtgggtgtgtttgctgccctgcacctcacagcgctcttccttactctgtacgaatggcggagtccctacgggctcacgccacgcggccgcaaccgtggtaccgtcttctcctactcctccgctctcaacctctgctacgccattctcttcggacgcactgtctccagtaagacacccaagtgtcctaccggacgcttcctcatgaatctctgggcaatcttctgcctgctggtgctatccagttacacagccaacctggcagctgtcatggtcggggacaagacatttgaggagctgtctggaatccatgatcccaagctgcaccacccttcccaaggcttccgctttggcaccgtgtgggagagcagcgcggaggcctacatcaaggcgagcttccccgagatgcacgcacacatgcgtcgccacagcgcacctaccactccacacggagtggccatgctcacgagcgacccgcccaagctcaacgccttcatcatggataaatcactactggattatgaggtgtccatagatgcggactgcaagctgctcaccgtgggcaaaccctttgcgatcgagggctacggcatagggctgccccaaaattcgccgctcacctccaacctgtcagagttcatcagtaggtacaagtcctcaggcttcattgatctgctccatgacaagtggtacaagatggtgccttgcgggaagcgggtgttcgccgtgacggagacgctgcagatgggggtctaccacttgtcagggttgtttgtcctgctgtgcctcgggctgggcagtgcacttctcacctcgctgggtgagcacgtcttctaccgcctggtgctgccgcgcatccgcaggggcaataagctgcagtattggcttcacacgagccagaagatccaccgagccctcaacacagggccaccagaggggcaacaggagagggcagagcaggagtgcagcggccccaaggaggagcaacctgcagccgacggtgcggggcgctggaggcgggtgcgccgggccgtggtggaacgggaacggcgcgtgcgtttcctgctggaacctggggaggctggcggggaccatccgtggctctgctccaatgggcccggggtgcaagcagaactgcgggagctggagctgcgcattgaggctgcacgggagcggctgcgtagtgcgctgctgcggcgaggggaactgcgggcccagcttggggatggcacccggctcaggccactgcgcctgctgcatgcggcgcccgccgagagctgaggaactacacggccacactgtccacgacagtttattctatatacaaacacgactctgtacactgcaattaaatagcgtggaacgtgaaaaaaa

SEQ ID NO: 2 - protein, 1003 aa, Mus musculus
MECVQTLWLSLALALARGSWVVRGHPQPCGVPTRAGASVRLAALLPRAPAARARVLAALATPSPRLPHNLSLELVAVASPTRDPASLARGLCQVLAPPGVVASITFPEARPELRLLQFLAAATETPVLSVLRREVRAPLGAPTPFHLQLDWASPLETILDVLVSLVRAHAWEDIALVLCRVRDPSGLVTLWTSRASQAPKFVLDLSQLDSGNDSLRATLALLGTLEGGGTPVSAAVLLGCSTAHAHEVLEAAPPGPQWLLGTPLPAEALPKTGLPPGVLVLGETGQPSLEAAVHDMVELVARALSSMALMHPERALLPAAVNCEDLKTGGSESTARTLARFLSNTSFQGRTGAVWVAGSSQVHVSRHFKVWSLRRDPLGAPAWATVGSWQDGQLDFQPGAAALRVPSPSGTQARPKLRVVTLVEHPFVFTRESDEDGQCPAGQLCLDPGTNDSARLDALFTALENGSVPRTLRRCCYGYCIDLLERLAEDLAFDFELYIVGDGKYGALRDGRWTGLVGDLLAGRAHMAVTSFSINSARSQVVDFTSPFFSTSLGIMVRTRDTASPIGAFMWPLHWSMWVGVFAALHLTALFLTLYEWRSPYGLTPRGRNRGTVFSYSSALNLCYAILFGRTVSSKTPKCPTGRFLMNLWAIFCLLVLSSYTANLAAVMVGDKTFEELSGIHDPKLHHPSQGFRFGTVWESSAEAYIKASFPEMHAHMRRHSAPTTPHGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMGVYHLSGLFVLLCLGLGSALLTSLGEHVFYRLVLPRIRRGNKLQYWLHTSQKIHRALNTGPPEGQQERAEQECSGPKEEQPAADGAGRWRRVRRAVVERERRVRFLLEPGEAGGDHPWLCSNGPGVQAELRELELRIEAARERLRSALLRRGELRAQLGDGTRLRPLRLLHAAPAES

SEQ ID NO: 3 - DNA, 2706 bp, Homo sapiens
atggagtttgtgcgggcgctgtggctgggcctggcgctggcgctggggccggggtccgcggggggccaccctcagccgtgcggcgtcctggcgcgcctcgggggctccgtgcgcctgggcgccctcctgccccgcgcgcctctcgcccgcgcccgcgcccgcgccgccctggcccgggccgccctggcgccgcggctgccgcacaacctgagcttggagctggtggtcgccgcgccccccgcccgcgaccccgcctcgctgacccgcggcctgtgccaggcgctggtgcctccgggcgtggcggccctgctcgcctttcccgaggctcggcccgagctgctgcagctgcacttcctggcggcggccaccgagacccccgtgctcagcctgctgcggcgggaggcgcgcgcgcccctcggagccccgaacccattccacctgcagctgcactgggccagccccctggagacgctgctggatgtgctggtggcggtgctgcaggcgcacgcctgggaagacgtcggcctggccctgtgccgcactcaggaccccggcggcctggtggccctctggacaagccgggctggccggcccccacagctggtcctggacctaagccggcgggacacgggagatgcaggactgcgggcacgcctggccccgatggcggcgccagtggggggtgaagcaccggtacccgcggcggtcctcctcggctgtgacatcgcccgtgcccgtcgggtgctggaggccgtacctcccggcccccactggctgttggggacaccactgccgcccaaggccctgcccaccgcggggctgccaccagggctgctggcgctgggcgaggtggcacgacccccgctggaggccgccatccatgacattgtgcaactggtggcccgggcgctgggcagtgcggcccaggtgcagccgaagcgagccctcctccccgccccggtcaactgcggggacctgcagccggccgggcccgagtccccggggcgcttcttggcacggttcctggccaacacgtccttccagggccgcacgggccccgtgtgggtgacaggcagctcccaggtacacatgtctcggcactttaaggtgtggagccttcgccgggacccacggggcgccccggcctgggccacggtgggcagctggcgggacggccagctggacttggaaccgggaggtgcctctgcacggcccccgcccccacagggtgcccaggtctggcccaagctgcgtgtggtaacgctgttggaacacccatttgtgtttgcccgtgatccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaagctgcaccacccggcgcagggcttccgcttcggcaccgtgtgggagagcagcgccgaggcgtacatcaagaagagcttccccgacatgcacgcacacatgcggcgccacagcgcgcccaccacgccccgcggcgtcgccatgctcacgagcgacccccccaagctcaacgccttcatcatggacaagtcgctcctggactacgaggtctccatcgacgccgactgcaaactgctgaccgtgggaaagcccttcgccattgagggctatgggatcggactgccccagaactcgccgctcacctccaacctgtccgagttcatcagccgctacaagtcctccggcttcatcgacctgctccacgacaagtggtacaagatggtgccttgcggcaagcgggtctttgcggttacagagaccctgcagatgagcatctaccacttcgcgggcctcttcgtgttgctgtgcctgggcctgggcagcgctctgctcagctcgctgggcgagcacgccttcttccgcctggcgctgccgcgcatccgcaaggggagcaggctgcagtactggctgcacaccagccagaaaatccaccgcgccctcaacacggagccaccagaggggtcgaaggaggagacggcagaggcggagcccaggtaa

SEQ ID NO: 4 - protein, 901 aa, Homo sapiens [sequence omitted]

SEQ ID NO: 5 - DNA, 1040 bp, Rattus norvegicus
ttgcggccgcgatgcttttttttttttttttttttttttacatgattgtcatcccagcattctggaggtagaggcaaaaggagcaggagctcaaggttacacacttaagttacttagttacatagcaagttcaaggttacacagttacttagtgatgtagcaagttcaagactaacctgagcagcaaaatactttcagaaagaaaggaagaaaggagggtatcggggagtcactgaacaccagtgagctgaaggcagggtaagacatagattcctttacagggtgggtggctgggactgtgcacacaaaggtggacagccctgacaggaaccacctcttcccagactccgttccatctgcagctggactgggctagtcccctggagaccatactggatgtgctggtgtccctggtacgggcacatgcctgggaggacattgctctagtactctgccgtgtccgggaccctggcggcctggtgacactctggactaaccatgctagccaggctccaaagtttgtgctggacctgagccggctggacagcaggaatgacagccttcgggctggactggccctgttgggggcgctggaaggagggggaaccccagtgcctgcagcagtcctcctaggctgcagcactgcccgtgcacatgaggtcctagaggctgcaccaccgggtccccagtggttgctgggcacacaattgcccgctgaggcactgcccacgactggtctgccacctggcgtgctggcgctgggggaaaccgaacaacactctctggaagctgtcgtccacgacatggtggagcttgtggctcaggcactcagtagcatggcccttgtacacccagagcgggcactgcttccagctgtggtgaactgtgatgacctgaaaacaggcggatctgaggcaacagggcgcaccttggctcggtgagtaggggctaacctaggggaaggcgtccagacccccagcctgacatcgtcctctgtgtgtcagtcaggtcatgagatctggccccgtgccgaattcttg

SEQ ID NO: 6 - DNA, 857 bp, Rattus norvegicus
ccgcaaccgtggcactgtcttctcttactcctccgcgctcaacctgtgctatgccattctctttggacgcactgtctccagtaagacgcccaagtgccctactggacgcttcctcatgaacctctgggcaatcttctgcctgctggtgctttccagttacacggccaacctggctgctgtcatggttggggacaaaacctttgaggagctgtctggaatccatgatcccaaggtgagggctggaaaaccgaaggggcttaaaaggctgtgctcagggtgggggtgaacactgggcccggcagaggcaggaccatctcctgcctgttcccttctcaacgagacttagtagcttaagtgcagtgctggcgaagaccagggtctgacgcctggtaggatccatagccccgccctgcccctatctctggcacttctctatttttgccacatccccagctccaatcacgaccacatccccattcttaagccttagaggtttgtttgttcgagctcagtctgtagaccaggctagcctcagactcaaagagacctgcctgcctctgcctcctgagtgctgagatttaaaggcgtgggcctaatctcatgactggccttagcaatcagtcctttaaaaactcccagcccccgcggcagggatgtcaggctgaagcacatctggatttattttcacctttgatggttttggagttcatgatagttctcacagcaacttccctctttgggatcaagctgctcatgtcctgctgacccccacccagccttgtgtcccttgtacctcggtgttaccacaagtggttgatggctgtggccaggtgaggactcctctgccgaattcttgg

SEQ ID NO: 7 - DNA, 736 bp, Rattus norvegicus
taaaattaaccctcactaaagggaataagcttgcggccgcaaccgtggcactgtcttctcttactcctccgcgctcaacctgtgctatgccattctctttggacgcactgtctccagtaagacgcccaagtgccctactggacgcttcctcatgaacctctgggcaatcttctgcctgctggtgctttccagttacacggccaacctggctgctgtcatggttggggacaaaacctttgaggagctgtctggaatccatgatcccaaggtgagggctggaaaaccgaaggggcttaaaaggctgtgctcagggtgggggtgaacactgggcccggcagaggcaggaccatctcctgcctgttcccttctcaacgagacttagtagcttaagtgcagtgctggcgaagaccagggtctgacgcctggtaggatccatagccccgccctgcccctatctctggcacttctctatttttgccacatccccagctccaatcacgaccacatccccattcttaagccttagaggtttgtttgttcgagctcagtctgtagaccaggctagcctcagactcaaagagacctgcctgcctctgcctcctgagtgctgagatttaaaggcgtgggcctaatctcatgactggccttagcaatcagtcctttaaaaactccccctcgtgccgaattcttggcctcgagggccaaattccctatagtgagtcgtattaaattcgta

SEQ ID NO: 8 - protein, 223 aa, Rattus norvegicus
IPLQGGWLGLCTQRWTALTGTTSSQTPFHLQLDWASPLETILDVLVSLVRAHAWEDIALVLCRVRDPGGLVTLWTNHASQAPKFVLDLSRLDSRNDSLRAGLALLGALEGGGTPVPAAVLLGCSTARAHEVLEAAPPGPQWLLGTQLPAEALPTTGLPPGVLALGETEQHSLEAVVHDMVELVAQALSSMALVHPERALLPAVVNCDDLKTGGSEATGRTLAR

SEQ ID NO: 9 - protein, 94 aa, Rattus norvegicus
RNRGTVFSYSSALNLCYAILFGRTVSSKTPKCPTGRFLMNLWAIFCLLVLSSYTANLAAVMVGDKTFEELSGIHDPKVRAGKPKGLKRLCSGWG

SEQ ID NO: 10 - protein, 102 aa, Rattus norvegicus
AQPFKPLRFSSPHLGIMDSRQLLKGFVPNHDSSQVGRVTGKHQQAEDCPEVHEEASSRALGRLTGDSASKENGIAQVERGGVREDSATVAAASLFPLVRVNF

SEQ ID NO: 11 - DNA, 21 bp, Mus musculus
cctctataacctttcccgagg

SEQ ID NO: 12 - DNA, 21 bp, Mus musculus
ctagagcaatgtcctcccagg

SEQ ID NO: 13 - DNA, 36 bp, Mus musculus
gatcctcgagccatggagatcgcctacaagcgacac

SEQ ID NO: 14 - DNA, 36 bp, Mus musculus
gatcggatccgcatgctcagctctccctatgacggg

SEQ ID NO: 15 - protein, 30 aa, Mus musculus
QSNEFGIFNSLWFSLGAFMQQGCDISPRSL

SEQ ID NO: 16 - protein, 30 aa, Mus musculus
STNEFGIFNSLWFSLGAFMRQGCDISPRSL

SEQ ID NO: 17 - protein, 30 aa, Mus musculus
VENNFTLLNSFWFGVGALMQQGSELMPKAL

SEQ ID NO: 18 - protein, 30 aa, Mus musculus
LVNQYSLGNSLWFPVGGFMQQGSTIAPRAL

SEQ ID NO: 19 - protein, 30 aa, Mus musculus
SMTSTTLYNSMWFVYGSFVQQGGEVPYTTL

SEQ ID NO: 20 - protein, 31 aa, Mus musculus
EEDALTLSSAMWFSWGVLLNSGIGEGAPRSF

SEQ ID NO: 21 - protein, 31 aa, Mus musculus
HGPSFTIGKAIWLLWGLVFNNSVPVQNPKGT

SEQ ID NO: 22 - protein, 31 aa, Mus musculus
RNKVFSFSSALNVCYALLFGRTAAIKPPKCW

SEQ ID NO: 23 - protein, 31 aa, Mus musculus
RGTVFSYSSALNLCYAILFGRTVSSKTPKCP